(19) United States
(12) Patent Application Publication
(10) Pub. No.: US 2010/0168532 A1
(43) Pub. Date: Jul. 1, 2010

(54) SYSTEMS AND METHODS FOR MEASURING BRAIN ACTIVITY

(75) Inventors: Allen E. Waziri, Denver, CO (US); Ronald G. Emerson, Chappaqua, NY (US); Guy M. MCKHANN, Bronxville, NY (US)

Correspondence Address: WilmerHale/Columbia University, 399 PARK AVENUE, NEW YORK, NY, 10022, US

(73) Assignee: The Trustees of Columbia University in the City of New York, New York, NY (US)

(21) Appl. No.: 12/606,174

(22) Filed: Oct. 26, 2009

Related U.S. Application Data
(60) Provisional application No. 61/108,438, filed on Oct. 24, 2008.
(60) Provisional application No. 61/181,986, filed on May 28, 2009.

(51) Int. Cl. A61B 5/0476; A61B 5/00

(52) U.S. Cl. 600/301; 600/544

(57) ABSTRACT

A device for measuring intracortical brain activity of a subject is provided. The device includes a support member configured for intracranial insertion in the subject. The device comprises a plurality of electrodes are positioned in an electrode region of the support member, which is configured to traverse the cerebral cortex of the subject when the support member is inserted intracranially in the subject. In certain embodiments, the device further comprises drainage region positioned on the support member such that two or more electrodes in the electrode region contact the cerebral cortex of the subject when the drainage region of the support member is in fluid contact with cerebrospinal fluid in the brain ventricle of the subject. In certain embodiments, the device further comprises a detector or probe for monitoring an additional brain parameter.

### CROSS-REFERENCE TO RELATED APPLICATIONS

This application claims the benefit under 35 U.S.C. §119(e) of U.S. Patent Application Nos. 61/108,438, filed Oct. 24, 2008, and 61/181,986, filed May 28, 2009, which are both hereby incorporated by reference herein in their entireties.

### COPYRIGHT NOTICE

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

Patients with critical neurological injuries frequently undergo bedside insertion of external ventricular drains (EVDs) for emergent management of elevated intracranial pressure and drainage of cerebrospinal fluid. Insertion of EVDs is commonly performed at most neurosurgical centers and used for clinical management for a wide range of acute neurological diseases and injuries. Many patients with these conditions remain in comatose or stuporous states and are therefore difficult to monitor for changes in brain physiology or ongoing neurological injury that could potentially be prevented or reversed.

Electroencephalography (EEG) is used in these individuals to detect seizures (a sign of brain injury), monitor for decreases in blood flow to the brain, and titrate medications for the treatment of elevated intracranial pressure. Continuous EEG is used in patients with neurological injury to detect electrographic seizures and clinically important changes in brain function.

Traditional EEG, which relies on electrodes that are affixed to the scalp, suffers from poor spatial resolution and the recorded data can be contaminated by electrical or patient-related artifacts. These factors limit the ability to detect secondary brain injury and are obstacles for the effective use of quantitative EEG analysis or automated EEG alarm systems in patients with acute brain diseases.

Accordingly, it would be desirable to provide systems and methods for measuring brain activity and managing elevated intracranial pressure, and in particular, systems and methods for providing higher quality recording of cortical potentials and drainage of cerebrospinal fluid.

### SUMMARY

Provided herein are devices for measuring intracortical brain activity in a subject. In one embodiment, the device is configured to measure cortical activity in the brain of the subject. The cortical activity can be measured from a plurality of electrodes positioned on the device such that two or more of the electrodes are in contact with the cerebral cortex of the subject when the device is inserted intracranially. In one embodiment, at least one electrode of the plurality of electrodes is in contact with the white matter or the overlying subdural space when the device is inserted intracranially.

The device described herein can overcome limitations of EEG derived from scalp electrodes, providing improvements in sensitivity, decreased artifact, more localized/focal recording from brain tissue at risk for ongoing injury, clarification of equivocal scalp EEG patterns, improved interpretation of observed changes detected by other neuromonitoring devices, and increased detection of seizures in comatose subjects, or any combination thereof. The device described herein can also be used to identify worsening neurological injury in a more rapid fashion than concurrently placed invasive neuromonitoring devices. This advantage can be important for instituting timely and appropriate clinical interventions that prevent permanent brain injury. The device described herein can also be used for application of electroencephalography in any setting of brain injury, including by way of example, the emergency room, intensive care unit, or field emergencies.

The device includes a support member having an insertable end configured for intracranial insertion. The support member can extend from the scalp. The device includes a plurality of electrodes positioned in an electrode region of the support member. The electrode region is configured to traverse the cerebral cortex of a subject such that at least two electrodes are in contact with the cerebral cortex of the subject when the support member is inserted intracranially. The spacing of the electrodes along the length of the device allows for discrete, high-fidelity recording from multiple electrodes within the cerebral cortex.

The device intracortical brain activity described herein can be adapted to include one or more additional functions. For example, the device described herein can also be adapted to function as a multifunction combination extraventricular drainage (EVD) and brain activity measuring device for concurrently draining brain ventricular fluid and measuring brain activity in a subject. In one example, the multifunction combination EVD and brain activity measuring device is configured to drain cerebrospinal fluid (CSF) from a ventricle in the brain of a subject and to measure cortical activity in the brain of the subject. In one example, multifunction combination EVD and brain activity measuring device described herein affords ease of use and consistency with insertion because the positioning of the electrodes in the electrode region reduces the need for exact precision during device insertion while still providing transcortical placement of two or more electrodes when the distal region of the device is placed in a brain ventricle of a subject.

The device described herein can be adapted to include any number of additional probes or monitors known in the art, including, but not limited to, a cerebral blood flow monitor, an intraparenchymal probe, a fiber optic cable, a thermal diffusion monitor, an oxygen-sensitive probe, a catheter (e.g., a microdialysis catheter) or any combination thereof. Any of the forgoing adaptations can be implemented with any examples or embodiments of the devices described herein.

In one embodiment, the device is a device for measuring intracortical brain activity of a subject, comprising: (a) a support member comprising at least a proximal end, a distal end, and an electrode region, wherein the support member is configured for intracranial insertion in a subject; (b) a plurality of electrodes positioned in the electrode region of the support member, wherein the electrode region is configured to traverse the cerebral cortex of the subject when the support member is inserted intracranially in the subject such that two or more electrodes in the electrode region contact the cerebral cortex of the subject, and (c) a plurality of conductors, each conductor individually connected to a corresponding electrode of the plurality of electrodes.

In another embodiment, the device is a device for measuring intracortical brain activity of a subject, comprising: (a) a hollow support member having an interior surface and an exterior surface, the support member comprising at least a proximal end, a distal end, an electrode region and a drainage region, wherein the support member is configured for intracranial insertion in a subject; (b) one or more drainage holes in the drainage region, wherein the drainage holes provide fluid contact between the interior surface of the support member and the exterior surface of the support member, (c) a plurality of electrodes positioned in the electrode region of the support member, wherein the electrode region and the drainage region are positioned along the support member such that two or more electrodes in the electrode region contact the cerebral cortex of the subject when the drainage region of the support member is in fluid contact with cerebrospinal fluid in the brain ventricle of the subject, and (d) a plurality of conductors, each conductor individually connected to a corresponding electrode of the plurality of electrodes.

In one example, the conductors are electrically coupled to an external sensing unit. In another example, the conductors extend through the interior of the support member from each electrode to an external sensing unit. In yet another example, the conductors extend outside of the support member from each electrode to the external sensing unit. In still a further example, the sensing unit is an electroencephalograph (EEG) sensing unit. In yet another example, the sensing unit is an EEG amplification and recording system.

In one example, the support member has a substantially tubular cross section. In another example, the support member comprises silicone.

In one example, the electrode region is positioned from about 3.5 cm to about 0.1 cm from the distal end of the support member. In another example, the electrode region spans a dimension of about 1.9 cm along the length of the support member. In still a further example, the electrode region is positioned about 2 cm from the drainage region. In yet another example, the device comprises at least 2 electrodes in the electrode region, at least 3 electrodes in the electrode region, at least 4 electrodes in the electrode region, at least 5 electrodes in the electrode region, at least 6 electrodes in the electrode region, at least 7 electrodes in the electrode region, or at least 8 electrodes in the electrode region. In one example, the device comprises 8 electrodes in the electrode region. In yet another example, at least one electrode in the electrode region contacts a brain region other than the cerebral cortex when the device is inserted intracranially in the subject.

In one example, two or more of the electrodes in the electrode region are at about 0.1 mm to about 5 mm in width along the length of the support member. In another example, two or more of the electrodes in the electrode region are at about 1 mm in width along the length of the support member. In yet another example, the electrodes in the electrode region are spaced such that the inter-electrode distance from the center of one electrode to the center of an adjacent electrode is about 0.1 mm to about 5.0 mm. In still a further example, the electrodes in the electrode region are spaced such that the inter-electrode distance from the center of one electrode to the center of an adjacent electrode is 2 mm.

In one example, the device further comprises one or more probes for detecting an additional brain parameter. In another example, the probe is positioned on the support member and configured to contact the brain of the subject when the device is inserted intracranially. In one example, the probe is a cerebral blood flow probe, a thermal diffusion probe, a oxygen sensing probe, a catheter or any combination thereof. In another example, the catheter is a microdialysis catheter. In still a further example, the device further comprises one or more conductors that are individually connected to each probe and extend from probe to a recording system outside of the support member.

In another embodiment, the subject matter described herein relates to a method for measuring abnormal electrical activity in the cerebral cortex of a subject, the method comprising: (a) inserting the device described herein into the brain of the subject such that two or more of electrodes in the electrode region of the device are in physical contact with the cerebral cortex of the subject; (b) measuring the electrical activity values recorded from the two or more electrodes in the electrode region of the device in physical contact with the cerebral cortex of the subject; and (c) performing bi-polar referencing of the electrical activity values recorded from the two or more electrodes in the electrode region of the device in physical contact with the cerebral cortex of the subject.

In yet another embodiment, the subject matter described herein relates to a method for measuring abnormal electrical activity in the cerebral cortex of a subject, the method comprising: (a) inserting the device described herein into the brain of the subject such that the drainage region of the device is in fluid contact with cerebrospinal fluid of a brain ventricle of the subject and two or more of electrodes in the electrode region of the device are in physical contact with the cerebral cortex of the subject; (b) measuring the electrical activity values recorded from the two or more electrodes in the electrode region of the device in physical contact with the cerebral cortex of the subject; and (c) performing bi-polar referencing of the electrical activity values recorded from the two or more electrodes in the electrode region of the device in physical contact with the cerebral cortex of the subject.

In one example, the abnormal brain activity is an electrographic seizure, a periodic epileptiform discharge, suppression-burst activity, spontaneous variability, reactivity to external stimuli, a presence of stage II sleep transients, a state change, a stimulus-induced rhythmic, periodic or ictal discharges (SIRPIDs), or any combination thereof.

In still a further embodiment, the subject matter described herein relates to a bedside alarm system comprising (a) a monitor adapted to receive at least electroencephalogram signals as an input from the device of claim 1, 2 or 20 and to produce an output of numerical or graphical values indicative of intracortical brain activity of a subject; and (b) a signal processor connected to receive the output indicative of intracortical brain activity of the subject and programmed to analyze that output to detect a change in brain function; and (c) a feedback transducer connected to the signal processor so as to selectively produce an alarm signal to the subject in the event the signal processor detects onset of the change in brain function.

In one example, the electroencephalogram signal is a continuous electroencephalogram signal.

In another example, the change of brain function is a seizure in the subject, a change in blood flow in the brain of the subject, a change in intracranial pressure, or any combination thereof.

### BRIEF DESCRIPTION OF THE DRAWINGS

In the drawings,

FIG. 1 is an illustrative side elevation view of a device for EEG measurement and extraventricular drainage, according to some embodiments of the disclosed subject matter;

FIGS. 2A-C show the radiographic appearance of a transcortical multicontact electrode (TCME), according to some embodiments of the disclosed subject matter;

FIGS. 3A-C show abnormal brain electrical activity recorded with a TCME, according to some embodiments of the disclosed subject matter;

FIGS. 4A-D show tracings from scalp EEG compared to concurrently recorded EEG from the TCME, according to some embodiments of the disclosed subject matter;

FIGS. 5A-G show tracings from scalp EEG compared to concurrent TCME recordings from a 73 year old woman with a right MCA infarction, according to some embodiments of the disclosed subject matter;

FIGS. 6A-G show EEG, CSA (compressed spectral analysis) analysis of scalp, TCME recording and multimodality monitoring in a 70 year old woman with subarachnoid hemorrhage, sepsis, and systemic hypotension leading to secondary diffuse intracererbal infarction, according to some embodiments of the disclosed subject matter;

FIGS. 7A-B show representative EEG and TCME data from a patient with grade IV subarachnoid hemorrhage and sepsis, according to some embodiments of the disclosed subject matter;

FIGS. 8A-C show EEG and TCME data recorded from a patient with acute neurophysiological changes secondary to hemorrhagic conversion of a large right MCA infarction (neuromonitoring devices placed in right frontal region), according to some embodiments of the disclosed subject matter;

FIGS. 9A-C show EEG recordings from a traditional scalp montage as well as from a TCME (boxed), according to some embodiments of the disclosed subject matter;

FIGS. 10A-C show selected EEG and TCME recordings and CSA analysis from a 80 year old woman with Hunt/Hess grade III subarachnoid hemorrhage, according to some embodiments of the disclosed subject matter;

FIGS. 11A-C show EEG and TCME tracings of seizure activity in a 20 year old woman who suffered traumatic brain injury, according to some embodiments of the disclosed subject matter; and

FIGS. 12A-E show data recorded from a patient with acute neurophysiological changes secondary to hemorrhagic conversion of a large right MCA infarction, according to some embodiments of the disclosed subject matter.

### DETAILED DESCRIPTION

Electroencephalography (EEG) measures the summed activity of post-synaptic currents in the brain. An action potential in a pre-synaptic axon causes the release of a neurotransmitter into the synapse that diffuses across the synaptic cleft and binds to receptors in a post-synaptic dendrite, resulting in a flow of ions into or out of the dendrite, which in turn results in compensatory currents in the extracellular space. These extracellular currents generate EEG voltages and the activity measured by EEG is an aggregate of the electric voltage fields from millions of neurons.

Several methods for recording EEG signals are known in the art. For example, scalp EEG can be collected from tens to hundreds of electrodes positioned on different locations at the surface of the head. EEG signals (in the range of milli-volts) are amplified and digitalized for later processing and the data measured by the scalp EEG can be used for clinical and research purposes. Exemplary diagnostic applications of EEG include, but are not limited to, diagnosis, detection or treatment of epilepsy, sleep-related disorders, sensory deficits, brain tumors, general brain function and the like. In cognitive neuroscience, EEG can be used to investigate the neural correlates of mental activity from low-level perceptual and motor processes to higher-order cognition (for example, attention, memory, reading).

Traditional scalp EEG has poor spatial resolution and can be contaminated by artifact, limiting the utility of quantitative EEG analysis and the development of EEG-based alarm systems for patients. The device described herein can provide advantages over currently used devices including, but not limited to: (1) high quality intracranial EEG recording of subjects; (2) high quality intracranial EEG recording of subjects undergoing bedside EVD placement; (3) increased subject safety due to the intracranial implantation of a single device, rather than two independent devices (as is currently required for placement of both an EVD and a TCME); (4) decreased risk of brain injury and bleeding with device insertion; (5) decreased procedural time and complexity to permit widespread use of intracranial EEG recording in neurosurgical centers; and (6) reduced expense with regard to production or routine clinical use.

In one embodiment, the device described herein is a transcortical monitoring device that can be used for recording of electrical activity in the cerebral cortex of a subject. Using standard techniques for the placement of monitoring devices, insertion of the device described herein, will result in contact between two or more electrodes of the device and the cerebral cortex of the subject. Uses of the device described herein can include, but are not limited to monitor or detect a wide variety of neurological diseases and disorders including, but not limited to, stroke, seizure, intracranial hemorrhage and the effects of general anesthesia.

In another embodiment, the device described herein is a multifunction extraventricular drain and transcortical monitoring device that can be used for simultaneous bedside drainage of cerebral ventricular fluid and recording of electrical activity in the cerebral cortex of a subject. Current EVDs are designed for bedside insertion to a depth of 5-6 centimeters from the skull surface resulting in placement of the drain tip within the frontal portion of the lateral ventricle. Using standard techniques for the placement of monitoring devices, insertion of the device described herein, such that one of more drainage holes on the support member of the device are in fluid communication with cerebrospinal fluid (CSF) in the ventricle of a brain of the subject, will result in contact between two or more electrodes of the device and the cerebral cortex of the subject. In some examples, the spacing and positioning electrodes and drainage holes on the device can be increased or decreased depending on the brain structure of a particular subject.

The methods and devices described herein can be used on any subject. For example, the subject can be a subject suffering from acute neurological injury requiring insertion of an invasive monitoring device. Intracranial insertion of the device can be bedside insertion or it can be perioperative insertion. Any method known in the art can be used for insertion. For example standard pre-coronal burr holes can be fashioned with a twist-drill and electrodes can be inserted under direct vision, and then tunneled to a separate exit point. The transcortical position of the electrode can be confirmed via demonstration of phase reversal on EEG recording during insertion. Continuous data can be recorded from the device, along with data from traditional scalp EEG montage, using a bedside video EEG monitoring system.

The device described herein can be used to provide immediate access to intracranial EEG at the bedside for subjects with critical neurological injuries. In a further example, the device described herein can be used to provide immediate access to intracranial EEG at the bedside for subjects with critical neurological injuries requiring EVD insertion, as well as increased subject safety due to decreased complexity of insertion and lower likelihood of malfunction of individual components.

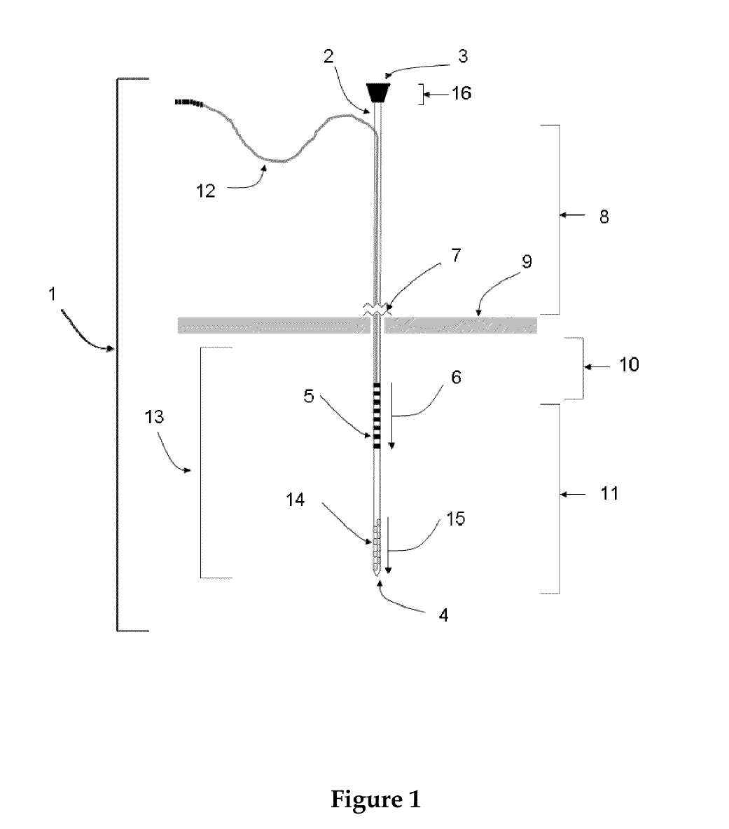

FIG. 1 is a schematic illustration of a multicontact intracranial device 1 in accordance with some embodiments of the disclosed subject matter. The schematic provided in FIG. 1 illustrates one embodiment of a multifunction extraventricular drain and transcortical monitoring device as describe herein, however other embodiments of the device, including embodiments which do not provide for a multifunction extraventricular drainage function are also suitable embodiments of the subject matter disclosed herein. In one example, the device 1 includes a hollow support member 2 having a proximal end 3 and a distal end 4, the support member being configured for intracranial insertion in a subject. The device includes a plurality of electrodes 5 positioned in an electrode region 6 of the support member. The electrode region is configured to traverse the cerebral cortex of the subject when the device is inserted through burr hole 7 of skull into the brain of the subject such that the external region 8 of the device is outside of the skull 9 of the subject and the subgaleal region 10 of the device and the intracranial region 11 of the device are inside the skull of the subject. The device includes a plurality of electrode connection conductors individually connected to corresponding ones of the electrodes. Each conductor can also be combined into a single cable or ribbon 12. The distal region 13 of the device has one or more drainage holes 14 in a drainage hole region 15 which provide openings for fluid to flow from a ventricle in the brain into the hollow support member of the device.

The device may employ one or more holes for fluid to flow from a ventricle in the brain into the hollow support member of the device in accordance with any one or more of the examples described herein. The drainage holes can be positioned in a drainage hole region having a length in the range of about 0.5 to about 3.5 cm in along the length of the support member in accordance with any one or more of the examples described herein, however the size and location of the drainage holes and of the drainage hole region can be adapted to a particular subject or to a particular application. The distance between the distal end of the drainage hole region can also be adapted to a particular subject or to a particular application. In one example, the distal end of the drainage hole region is located at the distal tip of the support member, however the device may comprise a drainage hole region wherein the distal end of the drainage hole region is located between about 0.5 to about 2.5 cm from the distal tip of the support member in accordance with any one or more of the examples described herein

When inserted intracranially in the subject, the proximal end of the device emerges from the burr hole and passes through the scalp, not shown, outside the body. At the proximal end, the support member can be adapted to make a fluid tight connection to a receptacle for accumulating fluid from the brain ventricle. The proximal end can comprise any type of valve 16 known in the art so as to provide fluid communication between the proximal end of the support member and a receptacle for accumulating fluid from the brain ventricle. The shape of the receptacle is not critical and can be of any shape or size. The device can be immobilized, for example by using an instrument immobilization device located at the burr hole entry in the subject's skull, to prevent or reduce shifting of the electrodes. Any instrument known in the art for immobilizing a device at a burr hole entry in a subject's skull can be used in conjunction with the device described herein.

The position of electrodes in the electrode region on the support member can be adapted to a particular subject or to a particular application. Using accepted and standard techniques the placement of monitoring devices, insertion of the device described herein will result in positioning of at least two electrodes within the cerebral cortex (e.g., grey matter of the brain). Intracranial insertion of the device can also result in positioning of one or more other electrodes in the underlying white matter and overlying subdural space. In one embodiment, electrodes contacting the white matter and/or the subdural space can be used to record a reference brain activity from a region outside of the cerebral cortex of the individual. The reference brain activity can be used as a baseline and/or to correct brain activity measured from the cerebral cortex of the subject.

The device described herein can comprise 2, 3, 4, 5, 6, 7, 8 or more than 8 electrodes positioned in the electrode region. The electrode region can be at least about in the range of about 0.1 cm to about 3.5 cm from the distal end of the support member. In one embodiment, the device can comprise a plurality of electrode contacts spanning a dimension of about 1.9 cm in length of the support member. The cerebral cortex of the brain is a region of tissue approximately 6-8 mm in thickness. Accordingly, in certain embodiments, the electrode region can span a length of at least about 0.5 cm along the length of the support member. The length of the electrode region can be modified according to the brain structure of a subject and according to the intended use of the device. The electrodes can also be placed along the entire inserted length of the support member, including at the distal end of the support member.

There is no specific limitation on the width and relative spacing of the individual electrodes, although the width and relative spacing can be optimized to allow for multiple electrode contacts spanning the cerebral cortex of a subject. The dimensions of the electrodes in the electrode region and can be modified according to the brain structure of a subject and according to the intended use of the device. In certain embodiments, the width of the electrodes in the electrode region can be in a range of about 0.1 mm to about 5 mm in width along the length of the support member. The inter-electrode distance between electrodes in the electrode region can be modified according to the brain structure of a subject and according to the intended use of the device. In certain embodiments, electrodes in the electrode region are spaced such that the inter-electrode distance from the center of one electrode to the center of an adjacent electrode is about 0.1 mm to about 5.0 mm. In one embodiment, the electrodes are 1 mm in width along the length of the support member and can be spaced apart along the length of the support member such that the inter-electrode distance from the center of one electrode to the center of an adjacent electrode is 2 mm.

The dimensions and relative positioning of the electrodes as well as the dimensions of the electrode region can be increased or decreased depending on the brain structure of a particular subject (e.g. the cortical width of a particular subject). Any method known in the art for determining cortex thickness, or for determining the relative position of the cortex to other structures within the brain can be used to position the device described herein or to adapt the relative positioning and dimensions of the electrodes on the support member of the device. Such methods include, but are not limited to magnetic resonance imaging, computerized axial tomography, radioactive neuroimaging, nuclear magnetic resonance, and the like.

In embodiments where the device is a multifunction extraventricular drain and transcortical monitoring device, the position of the electrodes in the electrode region can be configured such that at least two electrodes in the electrode region are in contact with the cerebral cortex of a subject when the drainage hole region at the distal tip of the support member is in fluid communication with a brain ventricle of the subject. The distance between the electrode region and the drainage region of the device can be variable and optimized for particular subjects or conditions. In one embodiment, the distance between the electrode region and the drainage region is sufficient to allow for device tunneling and external connection. For example, the spacing between the distal tip of the device can be increased or decreased depending on the brain structure of a particular subject.

The electrodes can comprise any electrically conductive material known in the art and can be similar to those utilized in current TCME devices. In one example, the electrode contacts are platinum electrode contacts.

Each of the electrodes in the electrode region can be electrically coupled to a sensing unit by means of a separate connection conductor (e.g., a conductive wire). The conductors can be run within (or outside) the support member of the device to external contacts designed for connection with any sensing unit known in the art, including, but not limited to an electroencephalograph (EEG) sensing unit, in accordance with any one or more of the examples described herein. Each of the electrode contacts can be capable of performing individual recording and signals that can be transmitted through a collected bundle of individual microconductors to a connection block, allowing for external interface with a standard EEG amplification and recording system. In one embodiment, each electrode is adapted to electrically communicate with the sensing unit on a dedicated channel. Suitable components for such a sensing unit are known to those skilled in the art of electroencephalography. The sensing unit can comprise a power supply and a controller. The power supply can be a primary battery, such as a rechargeable battery or other suitable device for storing electrical energy. The controller can include a processor, a memory, and a programmable computer medium. The controller, for example, can be a computer, and the programmable computer medium can be software loaded into the memory of the computer and/or hardware that performs requisite control functions.

The device described herein can also be in communication with a central processing unit (CPU), which has been programmed to run digital EEG or bipolar recording software in accord with the devices and methods described herein. The manner of connection can vary, and in one example the connection is made by a direct connect cable. Various other ways to connect the device to the CPU will be evident to those skilled in the art, including both wired and wireless. For example, the conductors individually connected to corresponding ones of the electrodes can be in electrical communication with a wireless transmitter, which is in turn in wireless communication with a receiver in electrical communication with a the CPU, permitting the CPU to be placed at some physical distance from the subject.

In one example, the device, the sensing unit, and the CPU have electronic components which can tolerate being continuously or almost continuously active. In one example, this feature can reduce the need to turn off or replace the device, the sensing unit or the CPU.

The support member of the device can include any material known in the art, including, but not limited to silicone or rubber. The support member can also include, or be coated with any biocompatible material known in the art. A “biocompatible” material as used herein, is a material which, when inserted into the brain of an individual, is tolerated by the individual's body and does not trigger major immune reactions or acute phase responses. Exemplary biocompatible materials suitable for use in the device described herein include materials having an inert surface, such as polymer materials (e.g. a polymer coated with a plurality of charged species (e.g. hydrophilic polyethylene glycols)), which prevent or reduce accumulation of biological materials on a surface over the course of a period of contact (e.g. hours, weeks, or months) between the biocompatible material and the biological material. Suitable biocompatible materials can also include, for example, carbon comprising inert materials such as those described in L A Thomson, F G Law, N Rushton, J Franks. Biomaterials 12, 37 (1991). Other materials suitable for coating the support member include, but are not limited to, ceramics (e.g., titanium nitride), turbostatic carbons (e.g., pyrolytic carbon), sputtered carbon coatings (e.g., Graphit-iC), Phosphatidyl choline di-ester, Teflon, or any combination thereof.

The support member can also be coated with an anti-clotting agent suitable for reducing or preventing the clotting of blood or other body fluid around the device. Exemplary anti-clotting agents suitable for coating the support member include, but are not limited to, heparin and tissue plasminogen activator (TPA), and other known anti-clotting agents.

Biocompatible coatings or anti-clotting coatings can be applied to the support member, for example, by bath, spraying, brushing, or dipping. In certain examples, the anti-clotting agent can be immobilized on the surface or it can be allowed to diffuse away from the member when it is inserted into the brain of a subject. In one example, the amount of an anti-clotting agent on the surface of the support member will be less than the amount required for treating a medical condition involving blood clots. One skilled in the art can readily determine the amount of an anti-clotting agent suitable for localized ant-clotting activity.

The length of the support member of the device can be of any length suitable for insertion into the brain of a subject. In certain examples, the length of the support member can be of any length between about 6 cm to about 90 cm. In one certain embodiments, the length of the support member is any length between the range of about 25 cm and about 35. The total length of the support member can also be divided into two or more individual segments. The support member can have a variety of cross-sectional shapes, including, but not limited to a flat shape, a planar shape, a U-shape, a V-shape or an O-shape.

In one embodiment, the support member of the device is not hollow. In another embodiment, the support member of the device is hollow. The external and internal diameter of the hollow support member can be adapted to a particular subject or to a particular application in accordance with any one or more of the examples described herein. In one example, the support member will have an outer diameter of about 3.0 mm. In one example, the inner diameter of the hollow support member has an inner diameter of 1.0 mm. The wall thickness for the hollow support member can be adapted to according to the chosen material for maintaining device integrity while allowing for maximization of internal diameter. In certain examples, the support member will have an inner diameter of about 1.0 mm to about 2.5 mm and an outer diameter of about 1.5 mm to about 3.0 mm or greater where the outer diameter of the support member is greater than the inner diameter of the support member by at least about 0.1 mm.

An application of the device described herein is bedside insertion of multiple electrode contacts across the cortical mantle in subjects. The device allows for several significant clinical advantages. The specific contact spacing allows for discrete, high-fidelity recording from multiple electrodes within the cerebral cortex. The array design affords considerable ease and consistency with insertion, as the locations and spacing of the electrode array does not require significant precision to achieve transcortical placement of several contacts, so that the catheter tip can be placed in the ventricle without difficulty. The ability to record from the electrodes during the insertion process allows for the detection of phase reversal and confirmation that the electrodes are indeed spanning the cortex. In one aspect, the device described herein can be used to perform bi-polar referencing between intracortical contacts, rather than referencing to a traditional scalp electrode reference. This feature can allow improvements in signal quality, averaging effects, and electrical noise level.

One application of the device described herein is continuous EEG monitoring. Continuous EEG monitoring can involve the use of a portable EEG machine connected to a subject to record seizure activity that is not apparent clinically (i.e., in the subject's mental status or by observing his/her movements). When subjects are put into medically-induced comas, the EEG pattern can be used as measure of depth of coma, and the medication can be titrated to an EEG end-point.

Brain activity recorded with the device described herein can be used for subjects with acute brain injury as well as for bedside EEG alarm systems. Because the device recordings can provide rapid or real-time detection of neuronal injury that would be detected in delayed fashion by other neuromonitoring modalities, the high-fidelity EEG data obtained via the device allows for optimization of bedside alarm systems to signal changes otherwise undetectable in subjects with critical neurological injuries. Thus in one aspect, the device described herein can be used as a subject monitor to detect changes in EEG recordings from the device.

For example, the device can be integrated with a bedside alarm system for continuous monitoring of subjects having critical neurological injuries to detect electrographic seizures, monitor for cerebral ischemia, and titrate medications for elevated intracranial pressure. Exemplary bedside alarm systems that can be used in conjunction with the device described herein include, but are not limited to those devices described in U.S. Patent Publication Nos. 2008/0287756; 2007/0191697; 2007/0191688; 2007/0129647; 2007/0024451; 2006/0235324; 2006/0155206; 2006/0149144; 2005/0240091; 2005/0062609; 2004/0111045; and 2002/0190863, each of which are incorporated by reference herein in their entireties.

Current procedures that require the placement of intracranial grids require that the procedure be performed in the operating room. One advantage of the device described herein is that the device can be inserted into the brain of a subject through the use of traditional methods at the bedside. For example, a subject having an aneurism may present with high intracranial pressure and or hydrocephalus. The device described herein can be inserted into the brain of a such a subject with, for example, a small burr hole to accommodate insertion of the device into the brain of the subject without transporting the subject to an operating room and the subject can be monitored using bedside transcortical monitoring.

Direct EEG recording from within the human cerebral cortex can provide advantages over recording from either scalp electrodes or non-cortical intracranial electrodes. As the electrical potentials recorded with EEG are generated by neurons resident within the cerebral cortex of the brain, a region of tissue approximately 6-8 mm in width at the brain surface, signal amplitudes recorded from this region are up to five times greater than potentials recorded from scalp electrodes or even the underlying white matter which is located millimeters away.

EEG recordings from tissues other than the cerebral cortex provide summed or averaged signals that are be generated by very large regions of brain tissue, thereby losing a great deal of specificity and sensitivity for recording asynchronous neuronal activity. Contacting multiple electrodes with the cerebral cortex provides higher quality EEG recordings decreases the average effect of each electrode and can yield a cleaner signal and a better signal to noise advantage thus permitting the detection of EEG abnormalities that are below the detection threshold of traditional scalp electrodes. In one example, the ability to record from the electrodes during the insertion process allows for the detection of phase reversal and confirmation that the electrodes span the cortex when the device is inserted into the brain of a subject.

Signal data from the subject obtained with the device described herein can be analyzed according to any method known in the art, including, but not limited to bipolar recording (e.g. recording where the potential difference between two electrodes is measured—see for example Fundamentals of EEG Technology: Basic concepts and methods By Fay S. Tyner, John Russell Knott, W. Brem Mayer, 1989). The ability to perform bi-polar recording between intracortical contacts, rather than referencing to a traditional scalp electrode reference, allows for improvements in signal quality, averaging effects, and electrical noise level. The use of bipolar recording allows for detailed measurement of potentials generated from brain tissue intervening between the two relevant contacts. Bipolar recording can also be used to determine position of the electrodes when the device is inserted into the brain of a subject, wherein the multiple electrode contact can be used as an internal reference.

For example, the signal data can be obtained from the subject at a desired sampling frequency, digitized, and filtered in the manner known to one of skill in the art and analysis can be performed using analysis techniques as described herein or according to any techniques known in the art. For example, E. Niedermeyer and F. Lopes da Silva (1999), Electroencephalography: Basic principles, clinical applications and related fields (4. Ed., Williams & Wilkins, Baltimore, USA) describes basic principles of electroencephalography.

Data fidelity obtained with the device described herein is improved over concurrent scalp EEG recordings, demonstrating an increase in signal amplitude (about 5 to about 10-fold). For example, epileptiform activity otherwise undetectable by scalp EEG can be detected with the device and methods described herein. Similarly, the devices and methods described herein enable detection of acute changes in EEG recordings from the device in isolation that are associated with a neurophysiological compromise in cases where the changes occurred up to six hours prior to the detection of any abnormalities with other concurrently placed neuromonitoring devices.

The device described herein also enables recording of ictal-appearing patterns that cannot be detected by traditional methods employing overlying scalp EEG. As such, recordings from the device can detect EEG changes associated with acute neurophysiological events that may not be detected by scalp EEG (e.g. secondary to increased signal amplitude, decreased artifact, and decreased signal averaging).

The device described herein can be further adapted to monitor one or more additional brain parameters or to provide additional therapeutic functions known in the art. In various examples, any type of tissue monitoring probe can be incorporated any embodiments of the device described herein. For example, the device can further comprise a detector or probe for monitoring an additional brain parameter positioned on the support member of the device. The positioning of electrodes relative to the positioning of the detector or probe for monitoring an additional brain parameter can be adapted to a particular subject or to a particular application. For example, certain probes will require contact with brain tissue to either monitor an addition brain parameter or to provide a therapeutic function. In such cases, the positioning of the electrode region on the support member of the device can be adapted such that intracranial insertion of the device suitable for monitoring the additional brain parameter, or for providing the therapeutic function, will result in contact of at least two electrodes are in the electrode region with the cerebral cortex of a subject.

In one embodiment, device is a multifunction combination extraventricular drainage (EVD) and brain activity measuring device further comprising a detector or probe for monitoring an additional brain parameter positioned on the support member of the device. The positioning of the detector or probe for monitoring an additional brain parameter may depend on the biological process or parameter being detected by the detector or probe for monitoring an additional brain parameter. Conductors from the detector or probe of the additional brain parameter can be run within (or outside) the support member of the device to external contacts designed for connection with any sensing unit for receiving a signal from the detector or probe of the additional brain parameter. The device may employ one or more the detector or probes of any number of additional brain parameters in accordance with any one or more of the examples described herein. Each of the additional detectors or probes of an additional brain parameter can be capable of performing individual recording and signals that can be transmitted through a collected bundle of individual microconductors to a connection block, allowing for external interface with a standard signal sensing units. In one embodiment, each detector or probe of an additional brain parameter is adapted to electrically communicate with the sensing unit on a dedicated channel. Suitable components for such sensing units are known to those skilled in the art of intracranial device insertion.

Any the device describe herein can be adapted to comprise and detector or probe for monitoring an additional brain parameter known in the art. For example, U.S. Pat. No. 5,916,171, which is hereby incorporated by reference herein in its entirety, discloses probes suitable for monitoring a number of brain tissue parameters, including DC potential, intracranial pressure (ICP), a single channel of EEG, blood flow and NADH fluorimetric. U.S. Pat. Nos. 4,784,150 and 4,945,896, which are both hereby incorporated in by reference herein in their entireties, describe probes for monitoring local cerebral blood flow and metabolic parameters including a laser doppler flow probe for measuring cerebral blood flow, and a probe for monitoring redox state (NADH). ICP can also be assessed using an extraventricular drain inserted into one of the lateral ventricles and connected to an external pressure transducer. ICP can also be measured using intraparenchymal probes. Such probes can comprise one or more strain gauge pressure sensors mounted at the tip of a thin catheter, or a fibreoptic catheter. Alternatively, subarachnoid, subdural and epidural devices can be used to measure ICP.

Cerebral blood flow (CBF) can be measured with thermal diffusion monitors to monitor focal cortical blood flow. For thermal diffusion monitoring, a probe (consists of two small gold plates, one of which is heated) can be inserted through a burr hole and placed on a cortical region of interest. Local blood flow can be calculated from the temperature difference between the two plates, which decreases with rising blood flow. This technique can also be modified by using an intraparenchymal probe which incorporates thermistors. U.S. Pat. No. 5,207,227, which is hereby incorporated by reference herein in its entirety, describes probes for monitoring local cerebral blood flow. U.S. Pat. No. 4,703,758, which is hereby incorporated by reference herein in its entirety, describes a probe for monitoring blood flow.

Devices for measuring brain oxygenation also exist in the art. For example, LICOX monitors measure oxygen tension as a measure of oxygen availability in the brain with oxygen-sensitive probes that can be inserted into specific locations of the brain to measure local or regional oxygenation, depending upon the insertion site. U.S. Pat. Nos. 5,876,577 and 6,144,866, which are hereby incorporated by reference herein in their entireties, describe probes for monitoring oxygen partial pressure.

Therapeutic devices for intracranial delivery of compounds to a subject are also known in the art, and the functions performed by these devices are also suitable for use with the methods and devices described herein. Exemplary devices of this type include microdialysis devices having at one end a catheter with permeable membrane capable of exchange fluids and solutes between the device and the brain of a subject upon insertion of the device into the brain of the subject.

The following example are set forth to aid in the understanding of the disclosed subject matter, and should not be construed to limit in any way the scope of the disclosed subject matter as defined in the claims which follow thereafter.

### EXAMPLES

### Example 1

### Neurophysiologic Monitoring in Patients

Neurophysiologic monitoring in patients with acute brain injury is a field of increasing focus and capability, providing opportunities for earlier and more appropriate therapeutic intervention in the neurological intensive care unit (NICU). The expanding array of relevant modalities includes non-invasive (e.g., transcranial Doppler ultrasound, scalp EEG) as well as invasive (e.g., brain tissue oxygen, cerebral microdialysis) techniques that monitor either upstream effectors or downstream indicators of neuronal health (Vespa, 2005).

The device described herein permits bedside placement of a transcortical “mini-depth” multicontact electrode (TCME), allowing for continuous monitoring of cortical potentials in critically ill patients with neurological injury.

Sixteen individuals admitted to the neurological intensive care unit with brain injury, requiring intracranial insertion of other invasive devices, were implanted with an eight contact TCME. Intracranial EEG was recorded and compared to concurrently recorded EEG from traditional scalp electrodes Immediate improvements in signal-to-noise ratio were noted in recordings from the TCME.

TCME recordings showed results in 12 patients, including evolving electrographic seizures (SZs, n=10), periodic epileptiform discharges but no seizures (PEDs, n=2), and acute changes related to secondary neurological injury (n=2). There was no scalp EEG correlation to these findings in 6 of 12 cases ( 5/10 with SZs, ½ with PEDs), and intermittent correlate in (all with SZs; ⅗ with only non-ictal appearing rhythmic delta on scalp). Two patients also exhibited rapid, prominent development of attenuation or suppression-burst patterns on TCME recordings without clear scalp EEG correlate, due to intracerebral hemorrhage or cerebral infarction. These TCME-specific abnormalities preceded detectable changes on neurological examination (6-10 hours) or in data recorded by other implanted neuromonitoring devices (2-8 hours). There were no definite adverse events associated with TCME insertion. The results described herein demonstrate that bedside insertion of a TCME, as a component of invasive neuromonitoring, can 1) safely provide high fidelity intracranial EEG recording in an ICU setting, 2) allow detection of ictal discharges that are not readily apparent on scalp EEG and clarify equivocal scalp EEG patterns, and 3) provide for real-time identification of abnormal brain activity associated with secondary neurological complications. Such TCME recordings can facilitate the development of EEG-based alarm systems in an ICU setting, help detect seizures and secondary cerebral injury as soon as they occur, and ultimately improve neurological outcomes.

The potential utility of continuous EEG recording (CEEG) for individuals with critical neurological injuries has been supported by studies from several groups demonstrating frequent nonconvulsive seizures or status epilepticus (NCSE) in this population (Towne, 2000; Claassen et al, 2005; Vespa et al, 2007). These clinically silent seizures identify a potentially treatable source of ongoing brain dysfunction and progressive injury. In addition, EEG has long been known to provide sensitive, and more importantly, real-time information regarding cerebral blood flow. Intraoperative EEG is widely used with carotid cross-clamping during endarterectomy to provide immediate information regarding the necessity for shunt insertion, with well characterized slowing and loss of alpha band power defining periods of critical ischemia (Sundt et al, 1974). Moreover, quantitative EEG (QEEG) has been shown to provide earlier detection of delayed ischemia in patients with subarachnoid hemorrhage (SAH) when compared to other modalities (Vespa et al, 1997; Claassen et al, 2004).

In spite of its potential benefits, use of conventional scalp electrode-derived EEG has limitations in the ICU setting. Interpretation of scalp EEG can be hampered by a poor signal to noise ratio, suboptimal long-term electrode contact with the scalp, interference or artifact from a wide variety of electrical devices involved with ICU care, and patient related factors (such as myogenic artifact) (Young and Campbell, 1999). In comatose patients, failure to detect or confirm underlying status epilepticus may contribute to depressed neurological function, secondary neuronal injury, and eventual poor outcome (Vespa et al, 1997; Claassen et al, 2004). These confounding factors (most notably artifact) have also precluded the development of practical bedside QEEG-driven alarm systems that could provide real-time information regarding ongoing or reversible neurological injury.

To determine whether a transcortical “mini-depth” multicontact electrode can overcome limitations of EEG derived from scalp electrodes and providing improvements in signal-to-noise ratio, decrease artifacts, localize recording from neuronal tissues at risk for ongoing injury, increase detection of nonconvulsive seizures when scalp EEG patterns are equivocal, and improve interpretation of observed changes in cerebral microdialysis, a series of patients admitted to neuro-ICU with a variety of neurological injuries requiring concurrent insertion of other standard invasive neurophysiological monitoring devices were examined.

A cohort of patients admitted to the NICU at Columbia University Medical Center between May 2006 and April 2008, harboring acute brain injuries and requiring bedside insertion of invasive therapeutic or monitoring devices, were studied. Patients were managed according to standard NICU protocols.

Commercially available eight-contact Spencer depth electrodes designed for clinical intracranial EEG recording with 2 mm (center-to-center) intercontact spacing and contact width of 1 mm, were chosen for use. As the width of the cortical mantle is roughly six to eight mm, this electrode design was selected to allow recording from 2-4 contacts within the gray matter as well as the positioning of several contacts within underlying white matter.

Electrode Localization and Surgical Procedure

TCME location was based on individual patient anatomy and pathology but, where possible, attempts were made to place the electrode in viable or penumbral tissue at maximal risk of secondary injury. A pre-coronal insertion site was identified, in most cases consistent with Kocher's point (approximately 11 to 12 cm posterior to the nasion and 2 to 3 cm lateral to the midline) to accommodate the concurrent placement of an EVD. The resulting cortical insertion site was located within the watershed zone between the anterior and middle cerebral artery vascular territories. In several cases of focal pathology, attempts were made to place devices in a perilesional or penumbral location.

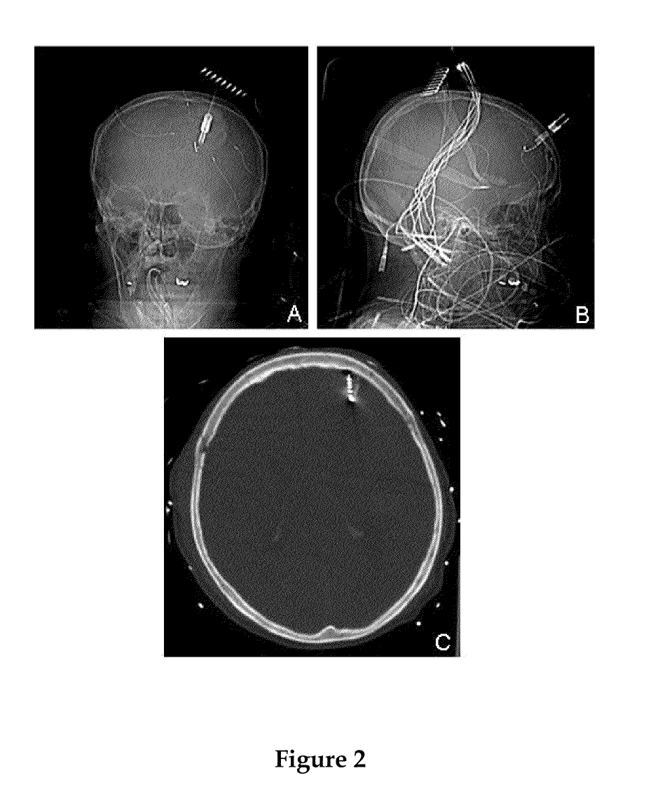

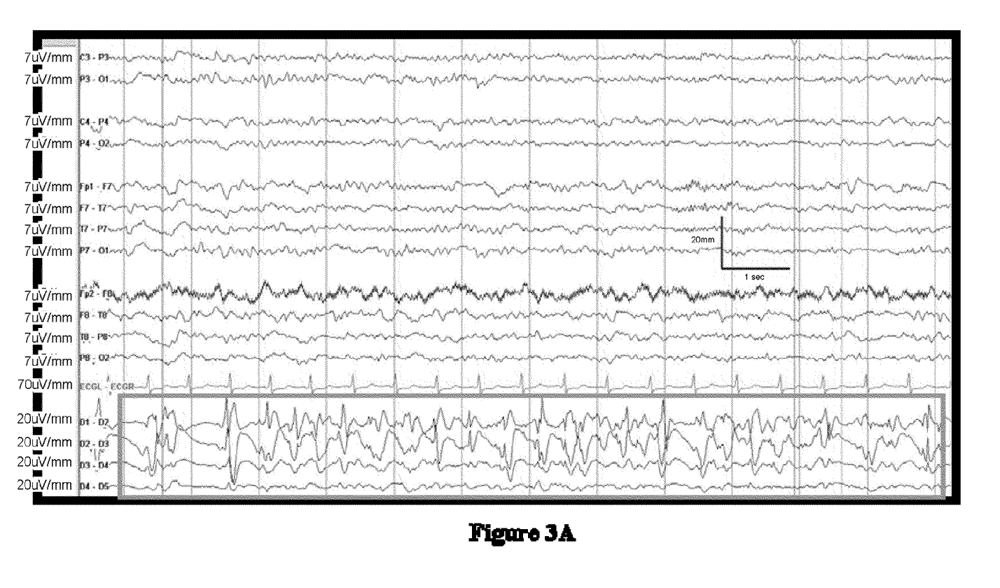

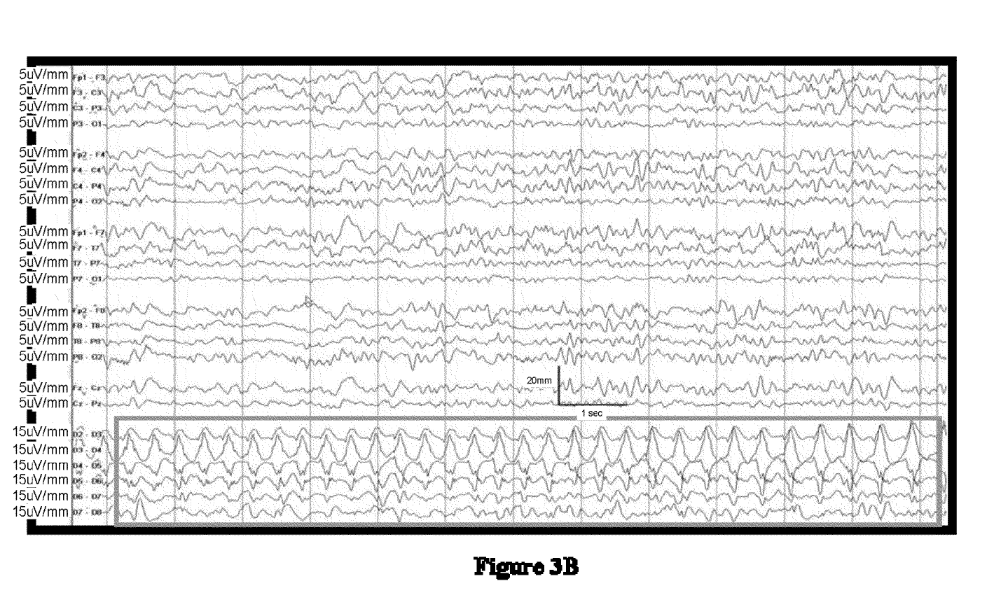

In most cases, two adjacent holes were drilled in the parasagittal plane approximately 0.5 cm apart. One burr hole was used for EVD placement and electrode insertion; when an EVD was not indicated the burr hole was used for the electrode in isolation. A small stab incision was fashioned lateral to the primary incision and the TCME was tunneled into the field. The electrode stylet was removed prior to insertion and the electrode was then passed to a transcortical position. During the latter half of the analysis, real-time recording from the electrode during insertion was utilized to confirm transcortical positioning. Insertion of other monitoring devices, typically including a Ventrix ICP monitor, microdialysis catheter, and Licox brain tissue oxygen monitor, was then accomplished through the remaining burr hole. During the initial analysis, the remaining monitoring devices were independently tunneled. A triple-lumen bolt device was used through the latter half of the analysis to reduce device movement. The wound was closed following final positioning of the monitoring devices. The devices were secured with stay sutures and sterile dressings were applied. A CT scan was also performed to confirm device location. FIGS. 2A-C show the radiographic appearance of the transcortical multicontact electrode (TCME), according to some embodiments of the disclosed subject matter. AP (FIG. 2A) and lateral (FIG. 2B) topograms demonstrate the typical appearance of the TCME associated with a neuromonitoring bolt, inserted in the left frontal region. FIG. 2C shows an axial CT image demonstrating the trans-cortical placement of the electrode.

Following the insertion of invasive monitoring devices, all patients had a full set of 21 standard scalp disk electrodes placed according to the International 10-20 system. In some patients, sterile subdermal needle electrodes were inserted around the surgical site in place of standard surface electrodes.

To determine if intracranial bleeding or infarction was associated with device placement on post-procedure scans, all implanted patients were assessed for evidence of cerebrospinal fluid leakage or infection associated with transcortical electrode insertion.

Continuous physiologic data from all monitoring devices were collected using an SQL database. Associated data from hourly neurological examinations and clinical interventions (e.g., vasoactive medications, sedative drips, etc.) were recorded using a bedside computer chart. EEG was recorded using a digital video EEG bedside monitoring system configured with a 200 Hz digital sampling rate. Quantitative EEG trending and analysis were performed using MagicMarker. Individualized quantitative EEG algorithms, including density spectral array (DSA), total power, and alpha/delta ratio, were developed for both surface electrodes and TCME. Recordings were reviewed multiple times daily with a final written interpretation by a board-certified electroencephalographer. Recordings were specifically evaluated for electrographic seizures (defined as lasting at least 10 seconds and showing clear evolution in frequency, morphology or location, or consisting of continuous epileptiform discharges reaching 3 Hz or faster); periodic epileptiform discharges (PEDs); suppression-burst activity; spontaneous variability; reactivity to external stimuli; presence of stage II sleep transients; state changes; and stimulus-induced rhythmic, periodic, or ictal discharges (SIRPIDs). Complete definitions of these EEG patterns have been previously described (Chong and Hirsch, 2005).

Results

Over 24 months, sixteen patients with acute brain injury underwent invasive neurophysiological monitoring including transcortical multicontact electrode placement. The sixteen individual cohort was composed from a group of thirteen women and three men ranging in age from 20 to 82 years (mean 61+/−19 years). The cohort included 10 patients with subarachnoid hemorrhage, 3 patients with traumatic brain injury, 2 with deep intracerebral hemorrhage, and 1 with embolic infarction (Table 1). Three of these patients had invasive monitoring devices placed in the operating room during emergent neurosurgical procedures, with the remaining thirteen undergoing bedside insertion. In patients with lateralized injury (N=3) monitoring devices were placed in the ipsilateral hemisphere. The side of insertion was determined based on individual

TABLE 1
Patient clinical data and details of intracranial monitoring

| | | | | Time to | | | |
|---|---|---|---|---|---|---|---|
| | | | | insertion | | | Monitoring |
| | | | | (days after | Devices | Device | duration |
| Patient | Age | Sex | Injury | admission) | placed | location | (days) |
| 1 | 81 | M | Traumatic | 0 | ICP, Licox, | R frontal | 3 |
| | | | SAH/SDH | | MD, TCME | | |
| 2 | 73 | F | R ICA occlusion | 4 | ICP, Licox, | R frontal | 6 |
| | | | | | MD, TCME | | |
| 3 | 54 | F | L temporo-parietal | 0 | ICP, TCME | *L parietal | 5 |
| | | | ICH | | | | |
| 4 | 61 | F | Grade V SAH | 7 | Licox, MD, | L frontal | 4 |
| | | | | | TCME, EVD | | |
| 5 | 40 | F | Grade IV SAH | 6 | ICP, Licox, | L frontal | 8 |
| | | | | | MD, TCME, | | |
| | | | | | EVD | | |
| 6 | 74 | F | Grade III SAH | 0 | TCME, EVD | *L frontal | 8 |
| 7 | 82 | F | Traumatic | 0 | ICP, Licox, | L frontal | 16 |
| | | | SAH/ICH | | MD, TCME | | |
| 8 | 45 | M | Grade IV SAH | 0 | Licox, TCME, | R frontal | 10 |
| | | | | | EVD | | |
| 9 | 73 | F | Grade III SAH | 0 | TCME, EVD | *L frontal | 7 |
| 10 | 70 | F | Grade IV SAH | 0 | ICP, Licox, | R frontal | 7 |
| | | | | | MD, TCME, | | |
| | | | | | EVD | | |
| 11 | 80 | F | Grade III SAH | 6 | ICP, Licox, | R frontal | 6 |
| | | | | | MD, TCME | | |
| 12 | 74 | F | Grade III SAH | 0 | ICP, Licox, | L frontal | 5 |
| | | | | | MD, TCME, | | |
| | | | | | EVD | | |
| 13 | 76 | F | IVH-hypertensive | 1 | ICP, Licox, | R frontal | 12 |
| | | | | | MD, TCME, | | |
| | | | | | EVD | | |
| 14 | 37 | F | Grade III SAH | 0 | ICP, Licox, | L frontal | 8 |
| | | | | | MD, TCME, | | |
| | | | | | EVD | | |
| 15 | 20 | F | Traumatic | 0 | ICP, Licox, | R frontal | 10 |
| | | | SAH/ICH | | MD, TCME | | |
| 16 | 38 | F | Grade IV SAH | 2 | ICP, Licox, | L frontal | 16 |
| | | | | | MD, | | |
| | | | | | TCMEx2, | | |
| | | | | | EVD | | |

As set forth in Table 1:
SAH—subarachnoid hemorrhage (with Hunt/Hess grade);
SDH—subdural hematoma;
ICA—internal cerebral artery;
ICH—intracerebral hemorrhage;
IVH—intraventricular hemorrhage;
SE—status epilepticus;
ICP—intracranial pressure monitor;
Licox-brain tissue oxygen tension monitor;
MD-cerebral (parenchymal) microdialysis catheter;
TCME—transcortical multicontact EEG electrode;
EVD—external ventricular drain;
*TCME inserted during operative prodecure

Three of these patients had invasive monitoring devices placed in the operating room during emergent neurosurgical procedures, with the other thirteen undergoing bedside insertion. In patients with well-lateralized injury (n=3), monitoring devices were placed in the ipsilateral hemisphere. The side of insertion was determined based on individual clinical factors (i.e. planned operative approaches, asymmetry of subarachnoid blood patterns, etc) for the remainder of the cohort. In 10 patients, the TCME was placed concurrently with and adjacent to an ICP monitor, Licox monitor, and microdialysis catheter. Ten patients had the electrode inserted on the day of presentation; the remaining 6 underwent delayed insertion of monitoring devices, with an average intervening time period of 4.3 (+/−2.4) days. The duration of monitoring ranged from 3 to 16 days (mean 7.7 days). There were no adverse events associated with device insertion, including hemorrhage, stroke, infection, or cerebrospinal fluid leakage. One patient had delayed hemorrhagic conversion of a large ischemic infarct near the insertion site which was unrelated to the insertion of monitoring devices.

TCME Data Quality

Intracortical recording was typically observed from three to four of the eight electrode contacts. Comparative analysis with concurrently acquired scalp EEG demonstrated higher signal amplitude, ranging from 2 to 5 fold, using bipolar recordings from TCME (despite the very small inter-contact distance). Potentials of lower amplitude were recorded from the most distal and proximal contacts of the transcortical electrode due to their positioning within the underlying white matter and overlying subdural spaces, respectively. In several cases, a shift over time in specific contacts providing the best data quality was noted. This effect was due to small movement of the electrode. In two of the early patients, the electrode was inadvertently dislodged during nursing care or transport for diagnostic studies. This technical issue was resolved with the incorporation of increased tunneling distance to the scalp exit site and the number of stay sutures used to secure the electrode described in the methods section below.

TCME Recording

Transcortical potentials were successfully recorded in all but two of the implanted patients. The first of these individuals, admitted with severe traumatic brain injury, entered barbiturate-induced electrocerebral silence (for treatment of medically refractory elevation of ICP) prior to implantation and subsequently suffered early brain death due to persistent ICP crisis and herniation. The second patient without successful recordings underwent electrode insertion during emergent decompressive hemicraniectomy; an immediate post-operative scan demonstrated that the electrode had been dislodged and was located within the subgaleal space, therefore providing no intracerebral contacts.

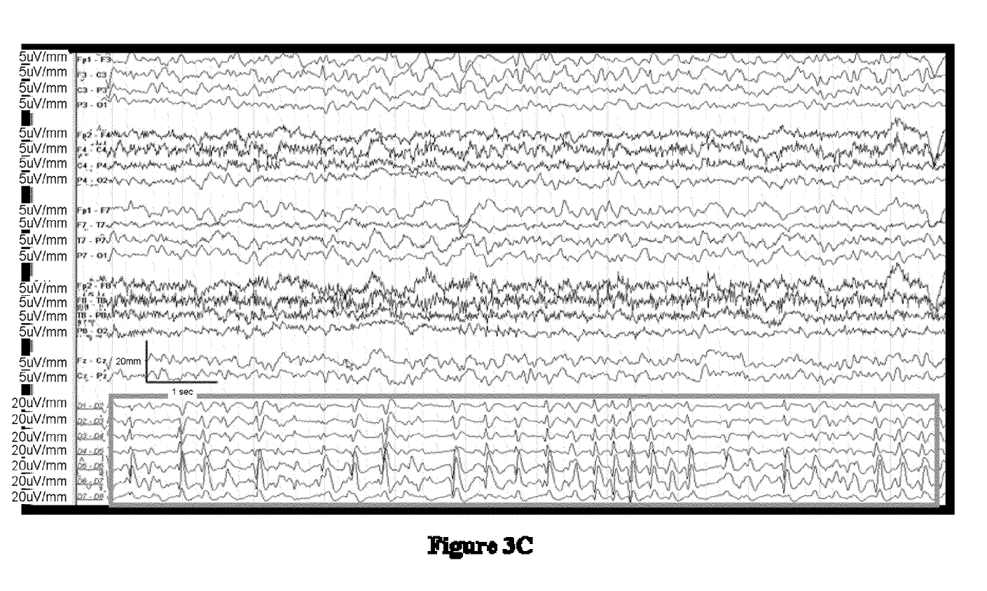

Analysis of TCME recordings from the remaining cohort of 14 patients identified 12 (86%) who had highly epileptiform findings (see Table 2 for summary), including evolving electrographic seizures (n=10; see FIG. 3) or periodic epileptiform discharges without seizures (PEDs; n=2). FIGS. 3A-C show abnormal brain electrical activity recorded with a TCME, according to some embodiments of the disclosed subject matter. Selected EEG recordings from three patients comparing traditional scalp EEG with concurrent TCME recordings (boxed), demonstrating electrographic seizure activity within TCME recordings without correlate in the overlying scalp EEG. Reference values (inset axes) indicate 20 mm tracing height and a 1 second interval; uV/mm are listed next to each channel (differs between intracranial vs. scalp channels). Filter settings for A, C: LFF 1 hz, HFF 70 Hz, notch off Filter settings for B: LFF I Hz, HFF off, notch off FIG. 3A is data recorded from a 70 year old woman with Hunt/Hess grade IV subarachnoid hemorrhage and a right frontal TCME. FIG. 3B is data recorded from a 74 year old woman with Hunt/Hess grade III subarachnoid hemorrhage with a left frontal TCME. FIG. 3C is data recorded form a 73 year old woman with Hunt/Hess grade III subarachnoid hemorrhage and a left frontal TCME, who demonstrated TCME-specific stimulus-induced rhythmic, periodic or ictal discharges (SIRPIDs). Two patients with seizures had stimulus-induced rhythmic, periodic, or ictal discharges (SIRPIDs) recorded by TCME without scalp EEG correlate (FIG. 3C).

TABLE 2
EEG abnormalities detected by the TCME and associated scalp EEG correlation

| | 1^{#} | 2 | 3* | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| Seizures from | ND | + | ND | + | − | − | + | − | +³ | + | + | + | + | − | + | + |
| TCME | | | | | | | | | | | | | | | | |
| Scalp correlate? | ND | − | ND | − | | | +² | | − | + | − | − | +/−⁵ | | − | +⁶ |
| PEDs from TCME | ND | − | ND | − | − | + | − | + | − | − | − | − | − | − | − | − |
| but no seizures | | | | | | | | | | | | | | | | |
| Scalp correlate? | ND | | ND | | | +¹ | | − | | | | | | | | |
| Abrupt EEG change | − | + | ND | − | − | − | − | − | − | + | − | − | − | − | − | − |
| assoc. with | | | | | | | | | | | | | | | | |
| secondary | | | | | | | | | | | | | | | | |
| neurological injury, | | | | | | | | | | | | | | | | |
| TCME | | | | | | | | | | | | | | | | |
| Scalp correlate? | | − | ND | | | | | | | +⁴ | | | | | | |

ND—no data
^{#}patient in barbiturate coma, suffered early brain death;
*TCME placed during emergent hemicraniectomy, dislodged from brain in immediate post-operative period;
¹Periodic epileptiform discharged (PEDs) seen by TCME correlated with probable non-convulsive status epilepticus on scalp recordings;
²patient with PEDs and occasional ictal runs on TCME, intermittent scalp correlate only clarified after comparison with TCME;
³Stimulus-induced rhythmic, periodic, or ictal discharges (SIRPIDs), detected by TCME only;
⁴appearance of attenuation in scalp EEG delayed by several hours;
⁵clear ictal activity from TCME without scalp correlate, but occasional late correlate with PEDs;
⁶intermittent low-amplitude delta activity in scalp EEG during TCME seizures

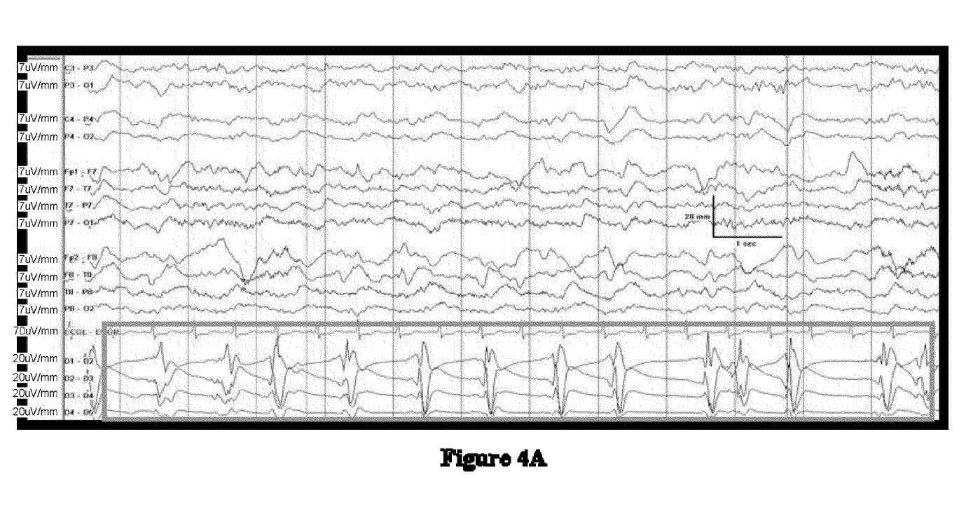

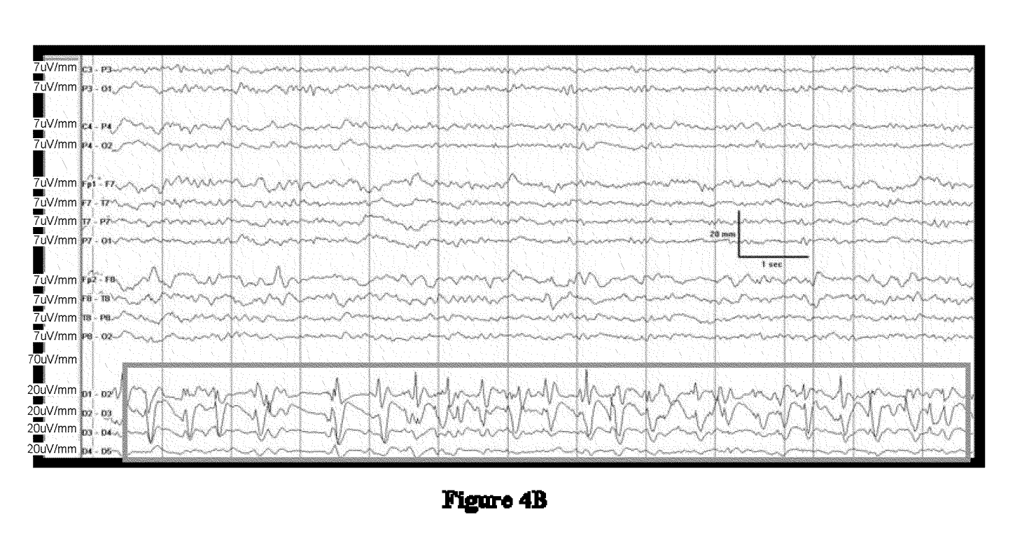

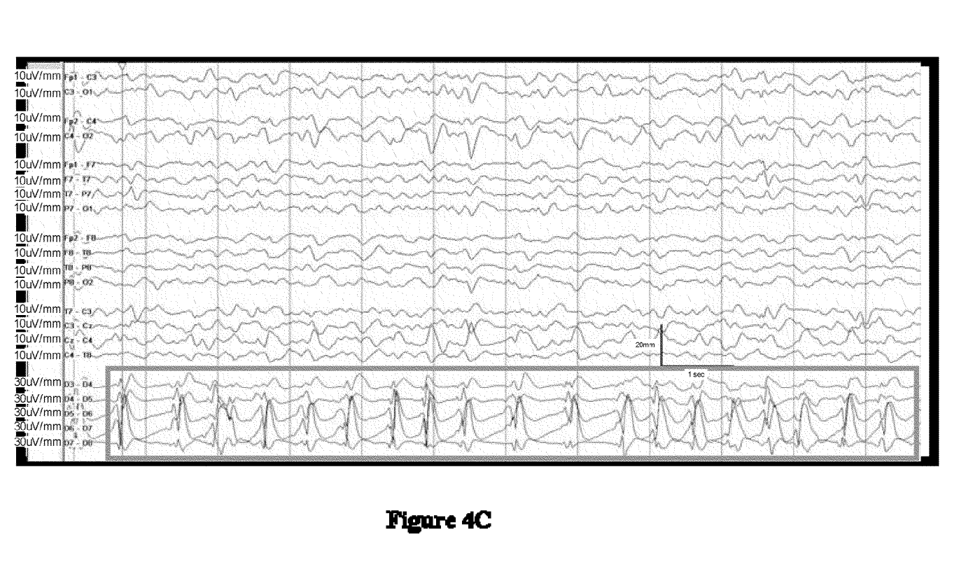

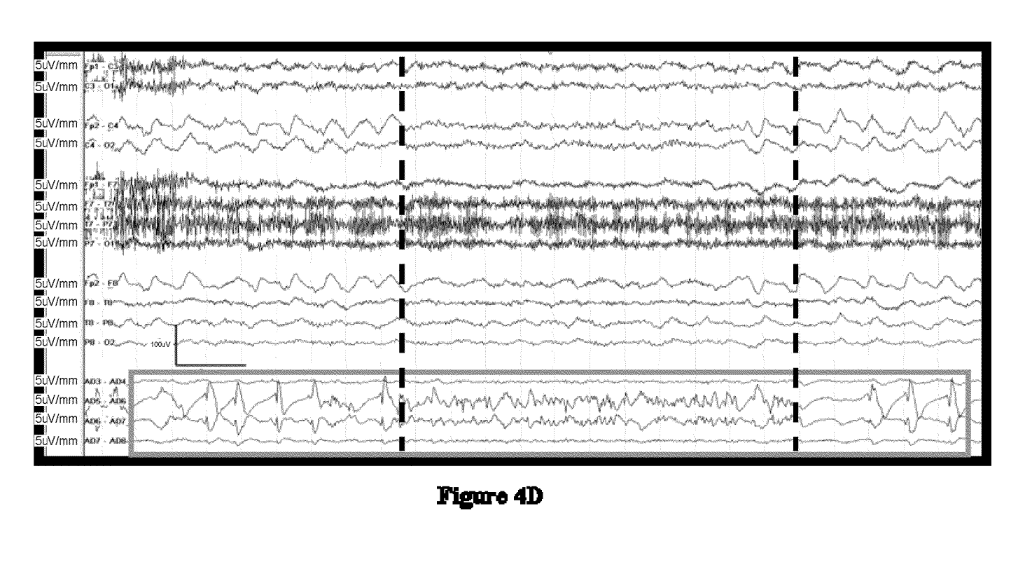

There was no scalp EEG correlate for many of the seizures or PEDs seen in TCME recordings. FIGS. 4A-D show tracings from scalp EEG are compared to concurrently recorded EEG from the TCME (boxed). Recordings from the TCME show scalp correlates and can be used to clarify equivocal scalp EEG patterns. Tracings from scalp EEG are compared to concurrently recorded EEG from the TCME (boxed). Reference axes: 20 mm tracing height, one second time interval. Filter settings (A/B/D): LFF1 Hz, HFF70 Hz, notch off; (C), LFF1 Hz, HFFoff, notch 60 Hz. FIGS. 4A and B are recordings from a 70 year old woman with Subarachnoid Hemorrhage (SAH) and a right frontal TCME. FIG. 4A demonstrates prominent periodic epileptiform discharges (PEDs) in TCME channels with corresponding semirhythmic delta activity (non-epileptiform/ictal-appearing) in the overlying scalp EEG. FIG. 4B demonstrates a period of evolving seizure activity in the TCME tracing from the same patient with pseudonormalization (resolution of semirhythmic delta) in the overlying scalp coverage. FIG. 4C displays recordings from a 76 year old woman with intraventricular hemorrhage (IVH) and a right frontal TCME, demonstrating prominent periodic epileptiform discharges at ˜2 Hz in TCME recordings and a vague semirhythmic correlate in the overlying scalp EEG. FIG. 4D provides compressed recordings from a 38 year old woman with subarachnoid hemorrhage and a right frontal TCME; reference x axis represents a 2 second time interval. An initial period of 2 Hz PEDs in the TCME tracing is associated with overlying rhythmic delta in the right hemisphere on scalp EEG. There is a subsequent period of lower voltage faster activity (e.g., ictal) lasting 11 seconds in the TCME tracings (following the first dashed line) with pseudonormalization in the overlying scalp EEG (resolution of rhythmic delta). Following this probable seizure in TCME recordings, the original pattern returns (following the second dashed line) with intracranial PEDs and extracranial rhythmic delta. Of the 10 patients with seizures in TCME recordings, 6 never showed a scalp correlate, 2 showed an intermittent scalp EEG pattern that could be considered potentially ictal (FIG. 4C), and 2 showed intermittent rhythmic delta only, without clear evolution and without a pattern that would traditionally be considered ictal (FIG. 4A, D). Of the 2 patients with PEDs but no seizures, one showed probable NCSE on scalp EEG, and one showed no scalp EEG correlate to the PEDs seen in TCME. Obvious clinical changes (e.g. worsening neurological examination or rhythmic motor activity) were not identified during episodes of seizures or PEDs recorded by TCME.

In several individuals, seizures detected by the TCME were associated with an increase in the local lactate to pyruvate ratio, determined via microdialysis catheter sampling; these data will be analyzed independently and reported in a future publication. A temporal relationship between device insertion and the onset of TCME-specific abnormal brain activity was not identified, indicating that recorded seizure activity was not attributable to insertion-related cortical injury.

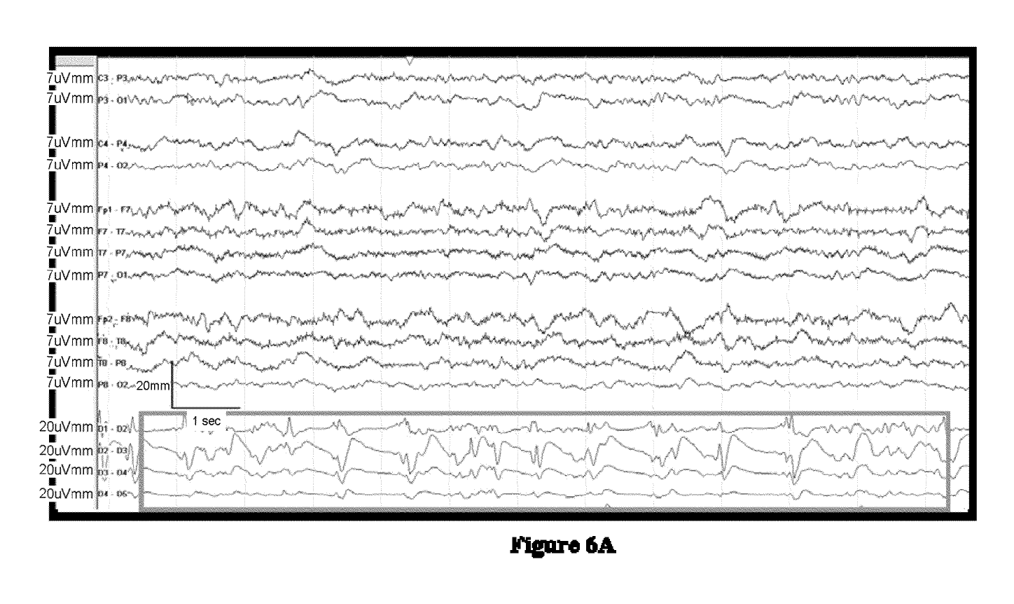

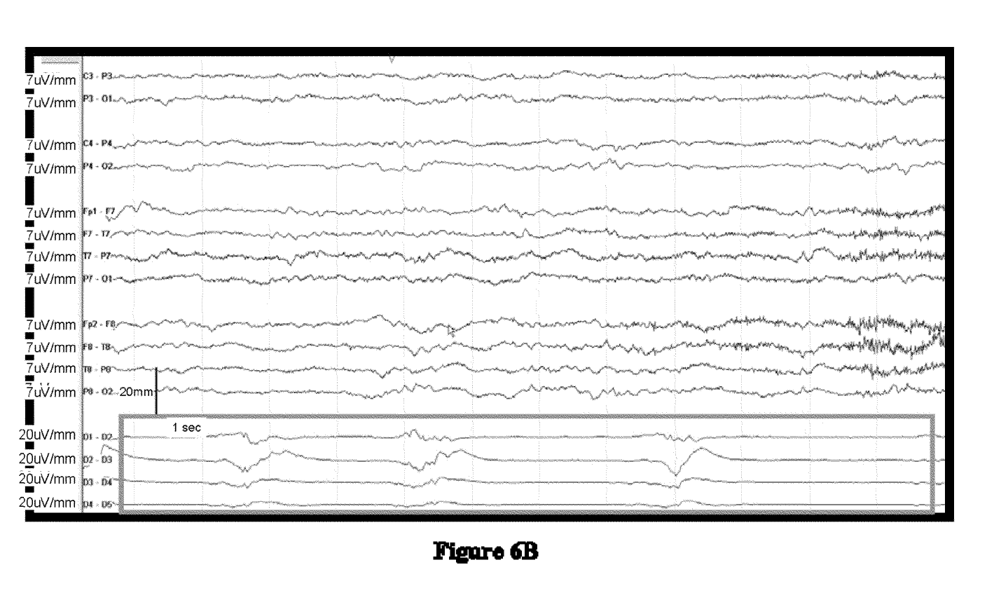

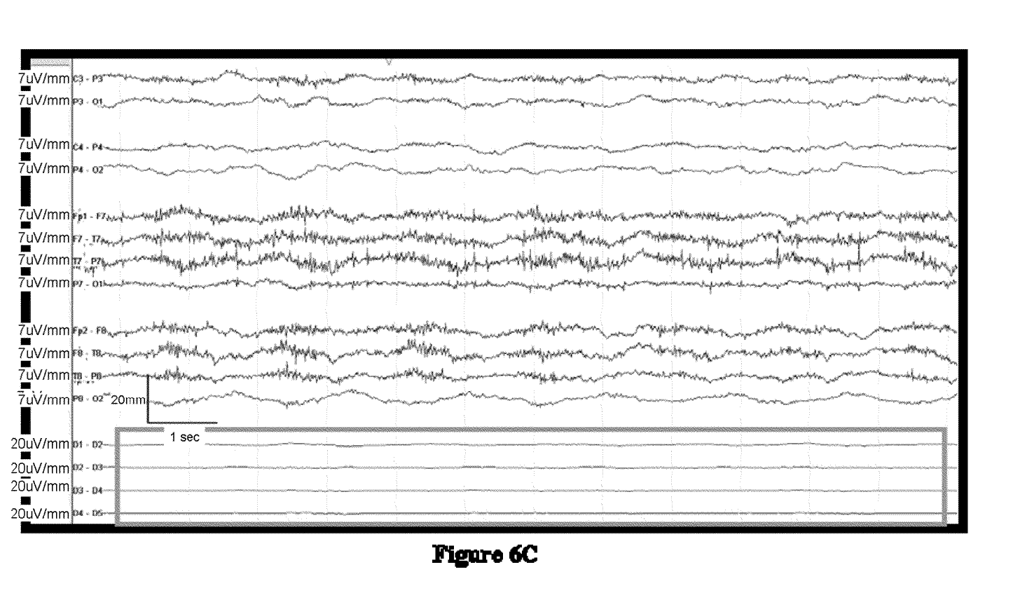

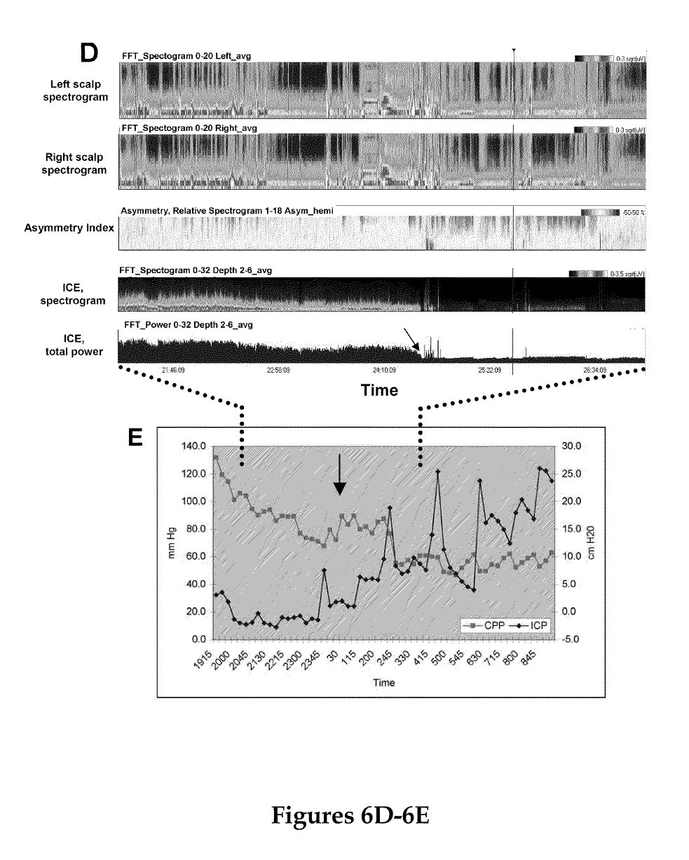

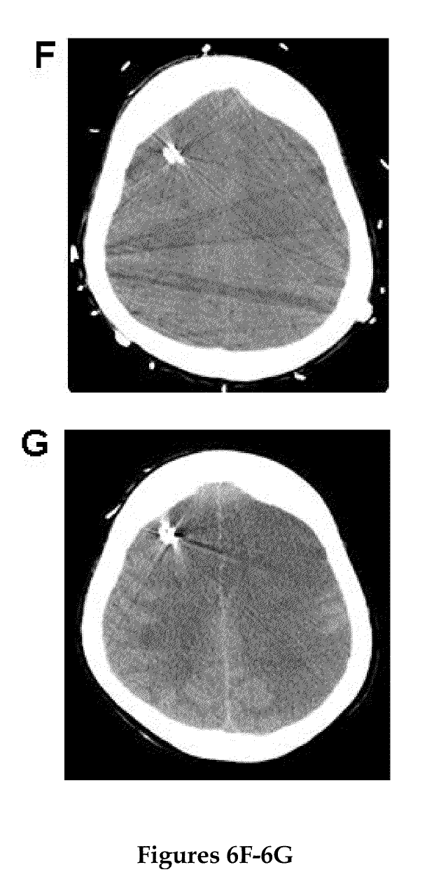

Well-defined increases in EEG power associated with TCME-specific seizure activity were visualized using QEEG trending techniques such as density spectral array (DSA). The quality of QEEG trending derived from TCME recorded EEG was considerably improved compared to QEEG trending performed with scalp-derived EEG, primarily due to increased signal amplitude and markedly decreased artifact (FIG. 6D).

Early Detection of Secondary Neurological Complications with TCME

Significant non-epileptiform, TCME-specific EEG changes were observed in two patients who suffered secondary neurological complications during the monitoring period. One patient with subarachnoid hemorrhage and underlying vasospasm developed widespread cerebral infarction following sepsis-associated hypoxia/hypotension, and the other developed hemorrhagic conversion of a large MCA infarction. In both patients, dramatic changes in TCME tracings (marked attenuation or suppression-burst patterns) appeared soon after the onset of secondary injury. These changes were not evident in simultaneous scalp EEG recordings (due to prominent muscle artifact on the scalp EEG or diminished signal amplitude). TCME-specific abnormalities preceded detection of concerning changes from other implanted neuromonitoring devices (by 2-6 hours) or changes in clinical exam (by >8 hours).

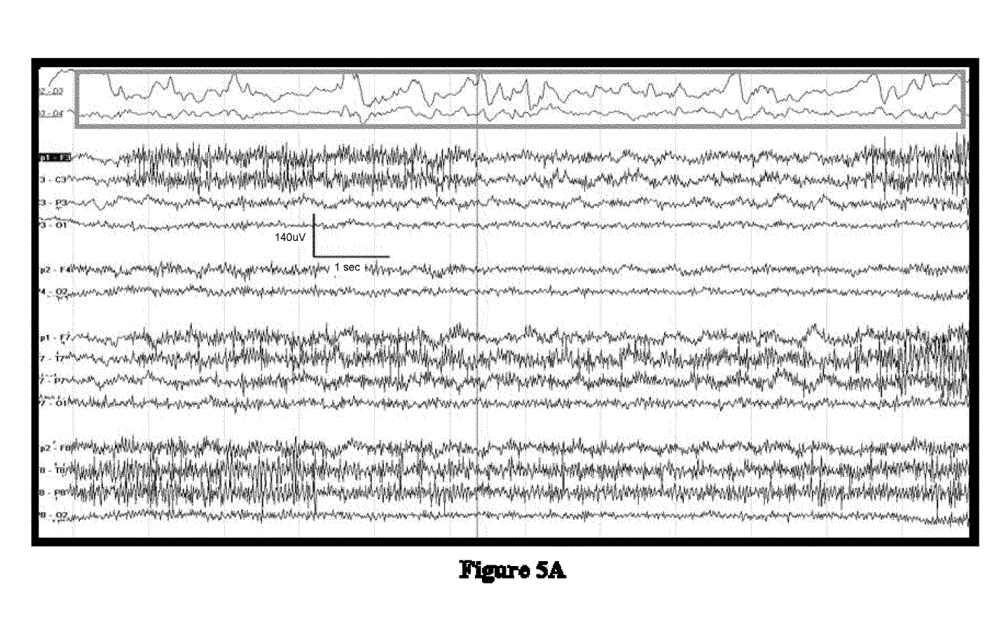

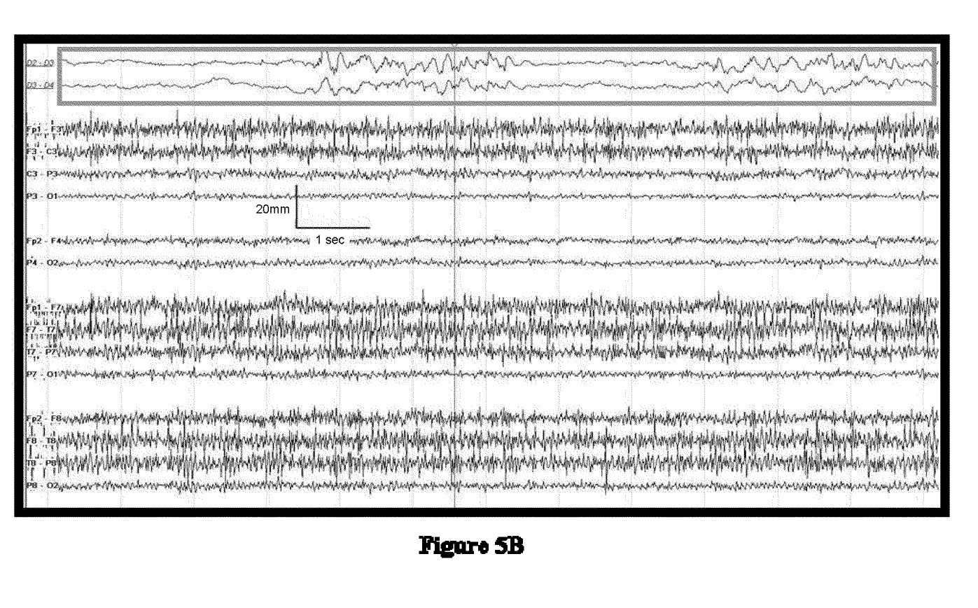

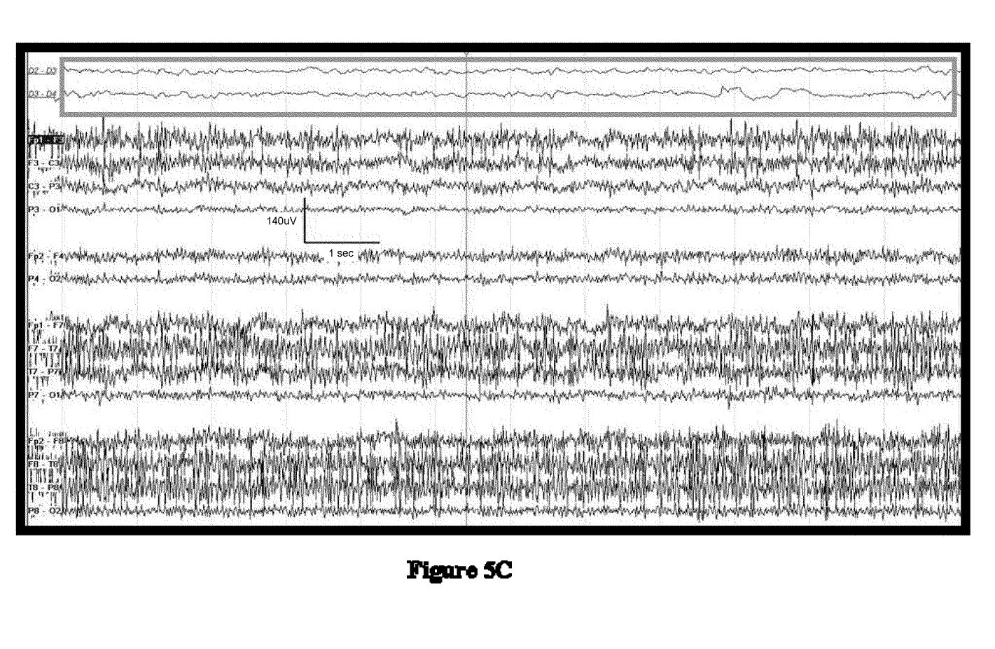

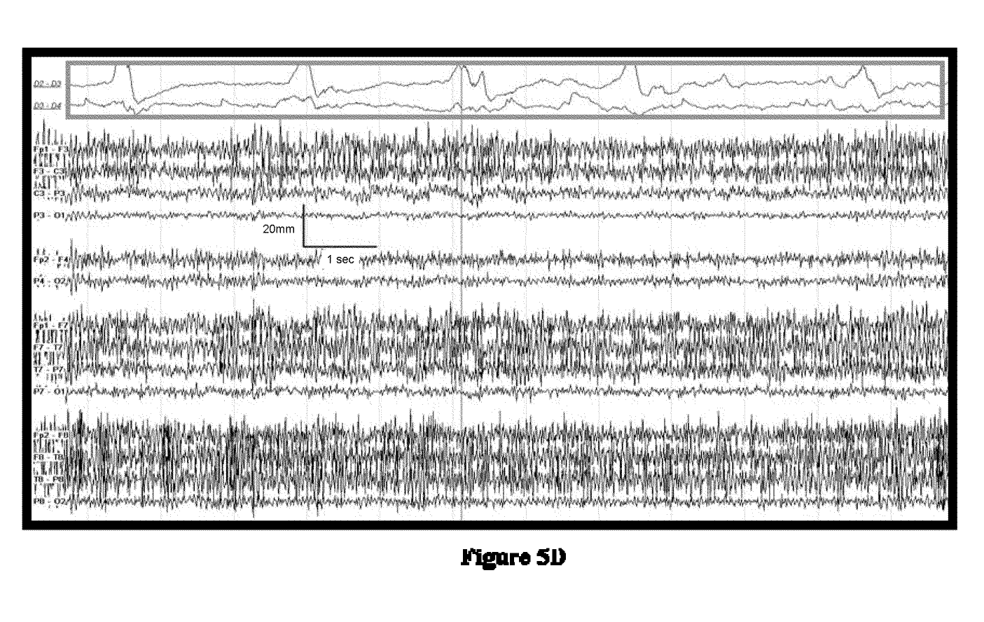

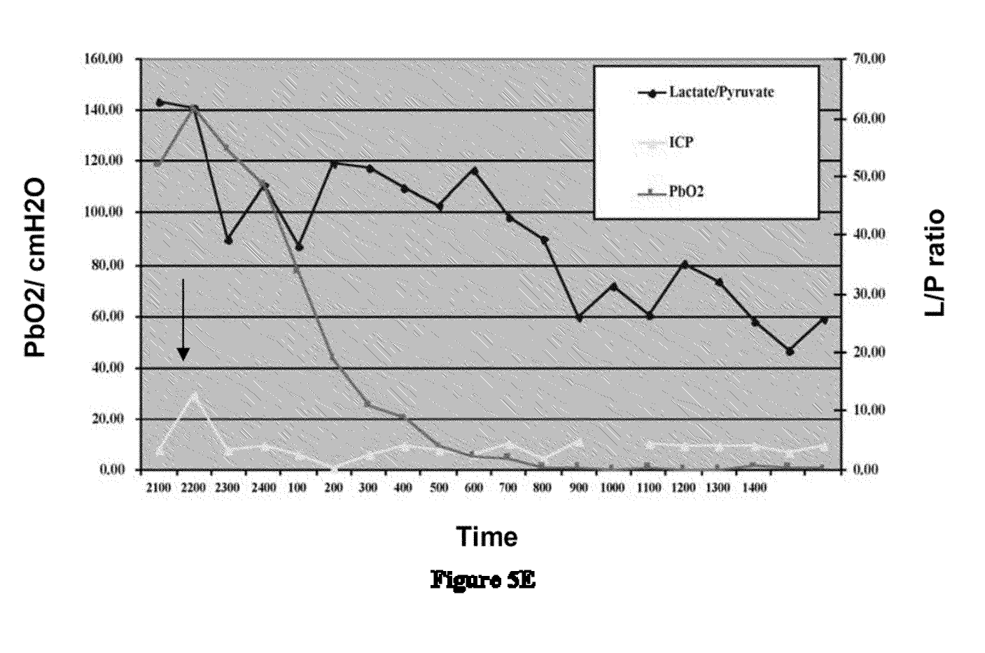

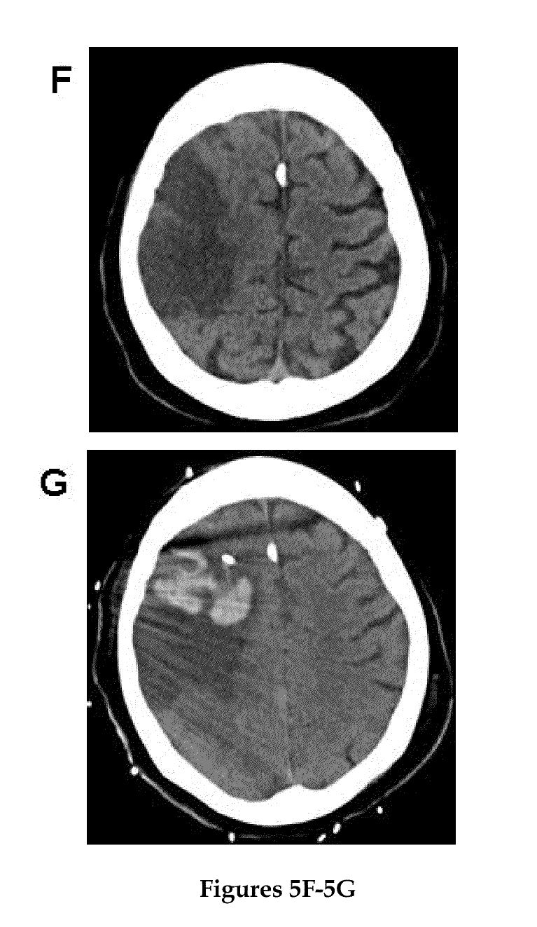

The first of these patients suffered a large right MCA territory infarction, prompting insertion of neuromonitoring devices within the right ACA/MCA watershed zone and therapeutic hypothermia. FIGS. 5A-G. show tracings from scalp EEG are compared to concurrent TCME recordings (boxed) from a 73 yo woman with a right MCA infarction. TCME inserted in the right frontal region provides early evidence for acute neurophysiological changes secondary to hemorrhagic conversion of a large right MCA infarction. Tracings from scalp EEG are compared to concurrent TCME recordings (boxed); reference axes indicate 20 mm tracing height, one second time interval. Scalp channels set at 7 uV/mm, TCME channels at 30 uV/mm. Filter settings: LFF1 Hz, HFF70 Hz, notch off. On day 4 following her stroke, rapid development of a suppression-burst pattern within EEG was recorded by TCME (FIGS. 5A-B). FIG. 5A demonstrates baseline EEG recordings; scalp EEG is uninterpretable due to myogenic artifact. FIG. 5B demonstrates a sudden conversion to a burst-suppression pattern at 9:45 pm recorded in isolation by the TCME with no interpretable change in the limited scalp EEG. Concurrently recorded scalp EEG was overwhelmingly contaminated by myogenic artifact (due to overt or micro-shivering from therapeutic hypothermia). The TCME-specific EEG abnormality evolved to a markedly attenuated state over several hours (FIG. 5C). FIG. 5C reveals a nearly isoelectric (flat) TCME recording by 4 am. During this time a gradual decline was also observed in the level of local cerebral oxygenation, detected by the implanted Licox sensor, which was declining two hours later but did not reach concerning levels until three hours following the onset of TCME-specific EEG changes. There was a small transient elevation in ICP at the presumed event onset, but no sustained elevation. By 6:30 am, cerebral activity returned in the TCME in the form of periodic delta waves at ˜0.5/second (FIG. 5D). Sequential evaluation of the local lactate/pyruvate ratio, sampled via microdialysis catheter, did not demonstrate evidence for a potentially concerning shift towards anaerobic metabolism, but rather remained markedly elevated throughout the monitoring period (FIG. 5E). Continuous (prior baseline) activity reappeared in the TCME by 7:30 am. Time-locked data from the other neuromonitoring devices including ICP monitor, Licox monitor, and microdialysis catheter are provided in FIG. 5E. The arrow demonstrates the time point equivalent to the onset of the burst-suppression pattern recorded by the TCME. Although a transient spike in ICP is demonstrated, intracranial pressure rapidly returned to baseline levels. No significant change in lactate/pyruvate level was detected by microdialysis (already markedly elevated prior to event). Although brain oxygenation decreased, as detected by the Licox monitor, the drop did not reach a concerning level until 2-3 hours after changes seen in the TCME recording. CT imaging before (FIG. 5F) and after (FIG. 5G) TCME-specific changes were detected demonstrates hemorrhagic transformation of the prior right MCA infarction. Detected changes in neurological exam did not occur until eight hours following the onset of EEG changes, at which time CT imaging demonstrated hemorrhagic conversion of her prior infarct associated with increased hemispheric mass effect (FIG. 5F, G).

The second patient was a 70 year old woman who suffered a Hunt/Hess grade IV subarachnoid hemorrhage from a ruptured anterior communicating artery aneurysm. Her course was complicated by the development of sepsis and hypotension requiring significant vasopressor support. FIGS. 6A-G show EEG, CSA analysis of scalp, TCME recording and multimodality monitoring in a 70 year old woman with subarachnoid hemorrhage, sepsis, and systemic hypotension leading to secondary diffuse intracererbal infarction. TCME provides the earliest indication of physiologic change. On day 6 following the insertion of neuromonitoring devices, QEEG obtained from TCME demonstrated a rapid and marked loss of power associated with the appearance of suppression-burst type activity in the raw EEG (FIGS. 6A-D). Similar changes were not visualized in EEG recordings from scalp electrodes. No immediate changes in neurological exam could be appreciated. FIGS. 6A-C demonstrate sequential raw EEG tracings comparing concurrently recorded scalp and TCME recordings (boxed); reference axes indicate 20 mm tracing height and a one second time interval. Filter settings: LFF 0.1 Hz, HFF 70 Hz, notch 60 Hz. An evolution can be seen from the highly epileptiform initial baseline EEG activity in the TCME (FIG. 6A) to a burst suppression pattern (FIG. 6B) and ultimately nearly complete attenuation (FIG. 6C), while the overlying scalp coverage did not demonstrate an obvious concerning change (although some degree of diffuse attenuation can be appreciated on these samples). FIG. 6D shows CSA analysis of scalp and TCME recordings in a 70 year old woman with Hunt/Hess grade IV subarachnoid hemorrhage, sepsis, and systemic hypotension. Shown is the quantitative EEG analysis of a 6 hour time period surrounding the identification of TCME-specific changes. The top 3 rows are derived from scalp EEG, and the bottom 2 from the TCME.

Six to eight hours following the onset of EEG changes recorded by TCME, a significant increase in intracranial pressure occurred (FIG. 6E). A significant drop in EEG power seen in isolation from the TCME was associated with a period of hypotension that led to global cerebral perfusion and infarction of multiple large vascular territories. This change preceded the associated increase in ICP by at least six hours. After a period of slow decrease, a significant and permanent drop in EEG total power was seen in isolation from the TCME (marked with arrow) with a similarly obvious and dramatic change in the TCME spectrogram. A similar trend could not be appreciated from the scalp-derived quantitative EEG trends. This event was associated with a period of progressive decrease in cerebral perfusion pressure (CPP) as well as a delayed and significant increase in ICP; the corresponding time interval is marked with dotted lines (FIG. 6E). CT imaging at that time demonstrated infarction of multiple large vascular territories, secondary to critical cerebral hypoperfusion in the setting of pre-existing vasospasm (FIG. 6F, G). CT imaging before (FIG. 6F) and after (FIG. 6G) the TCME-specific changes demonstrated infarction of bilateral anterior cerebral artery (ACA) and left middle cerebral artery (MCA) territories, secondary to hypoperfusion in the setting of pre-existing vasospasm. Review of the patient's vital signs demonstrated progressive systemic hypotension, with an associated decrease in cerebral perfusion pressure, which reached a nadir approximately one hour following the onset of TCME-recorded suppression-burst activity (FIG. 6E).

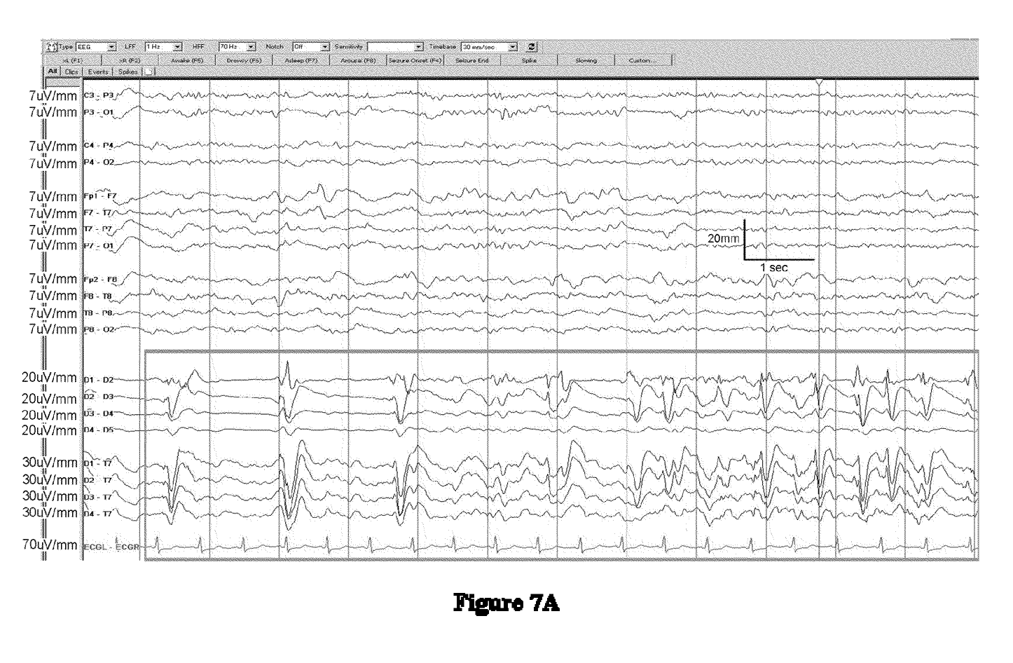

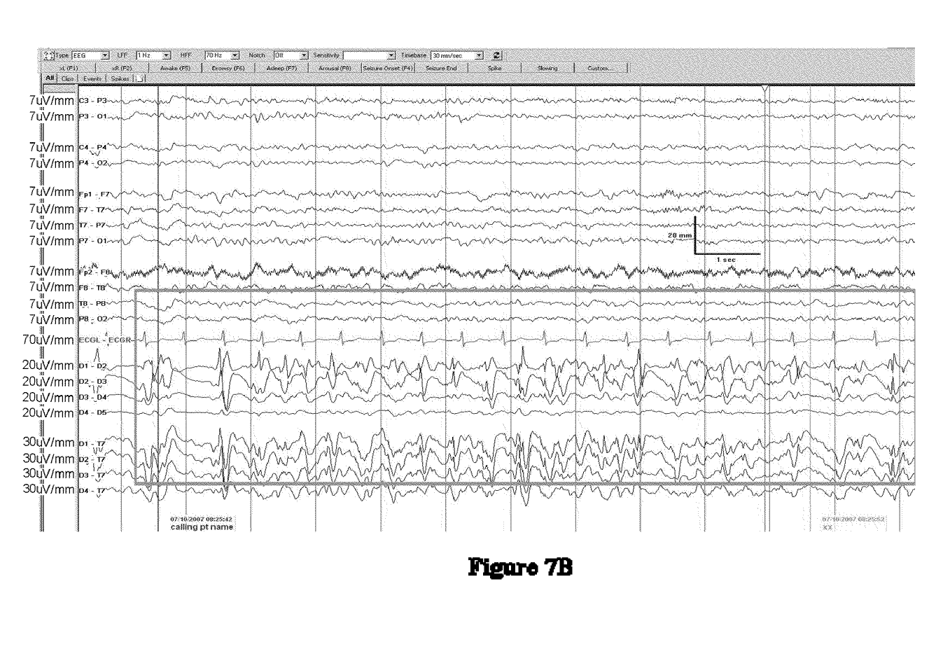

FIGS. 7A-B show representative EEG data from a patent with grade IV subarachnoid hemorrhage and sepsis. The data shown in FIG. 7A was recorded during a time period in which the patient became progressively less responsive; EEG from the TCME (boxed) demonstrates epileptiform discharges. Several hours later the patient became comatose with the TCME recording increasingly abnormal activity (FIG. 7B). Subsequent head CT demonstrated infarction in multiple vascular territories, secondary to hypoperfusion. Concerning findings from either ICP or Licox monitoring did not appear for 6-8 hours after the initial deterioration in neurological exam.

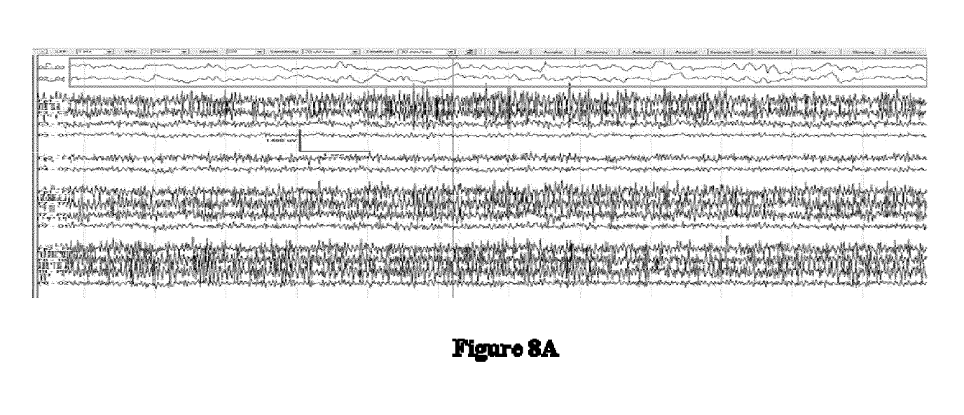

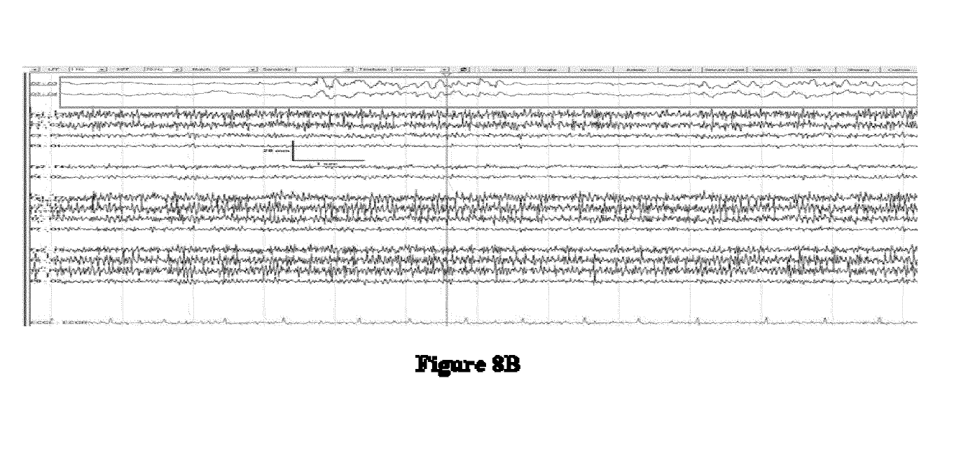

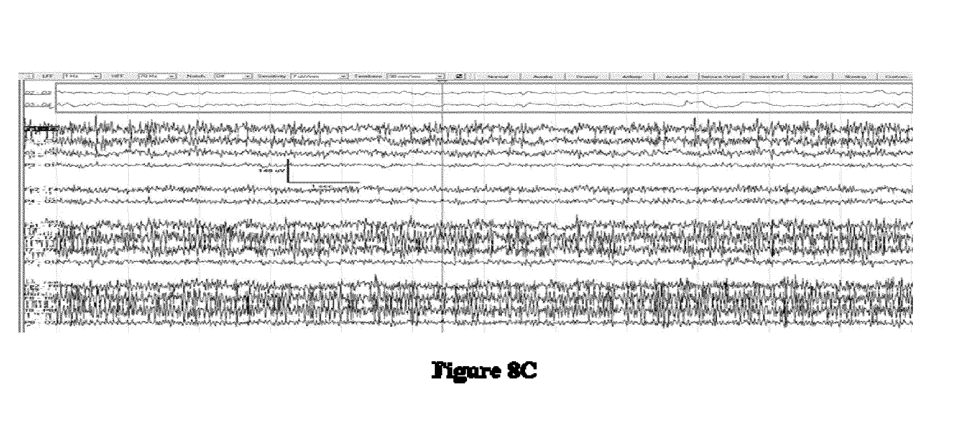

FIGS. 8A-C show EEG data recorded from a patient with acute neurophysiological changes secondary to hemorrhagic conversion of a large right MCA infarction (neuromonitoring devices placed in right frontal region). The patient did not exhibit a significant change in neurological exam until 6-9 hours after the detection of EEG changes. FIG. 8A shows baseline EEG (TCME data outlined in box); scalp EEG is unreadable secondary to muscle artifact from shivering. FIG. 8B demonstrates a sudden conversion to a burst-suppression pattern detectable by the TCME alone. FIG. 8C demonstrates further attenuation.

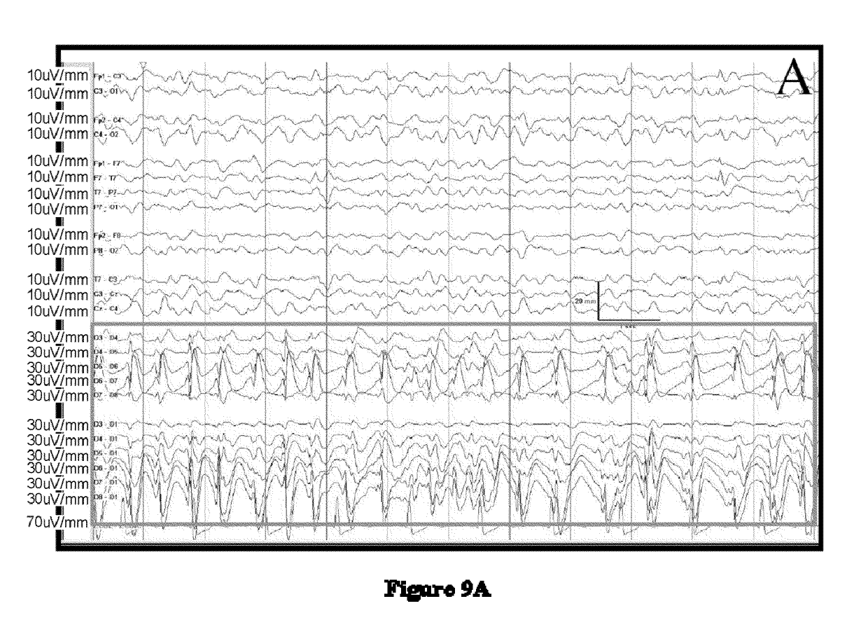

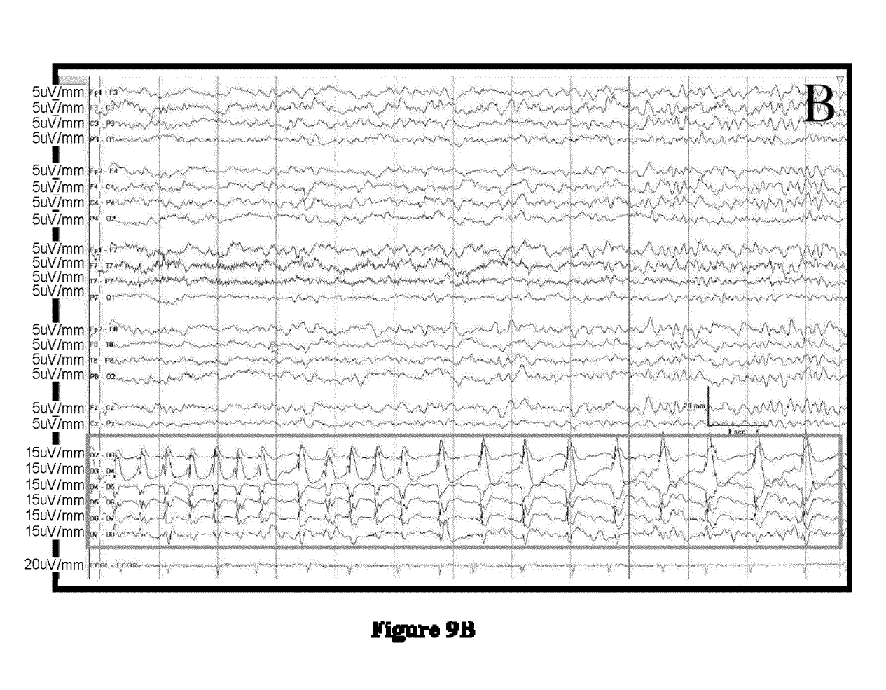

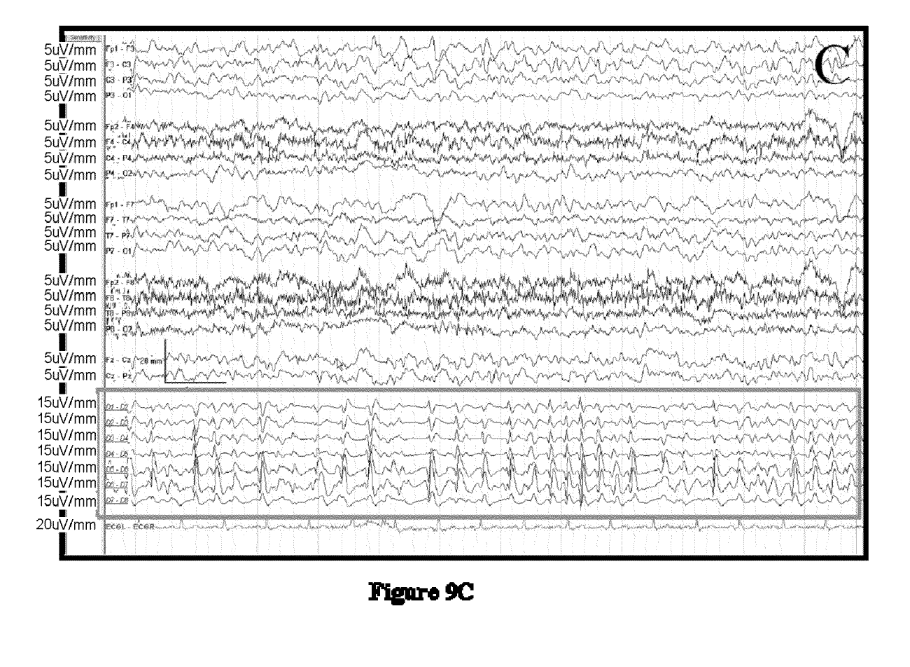

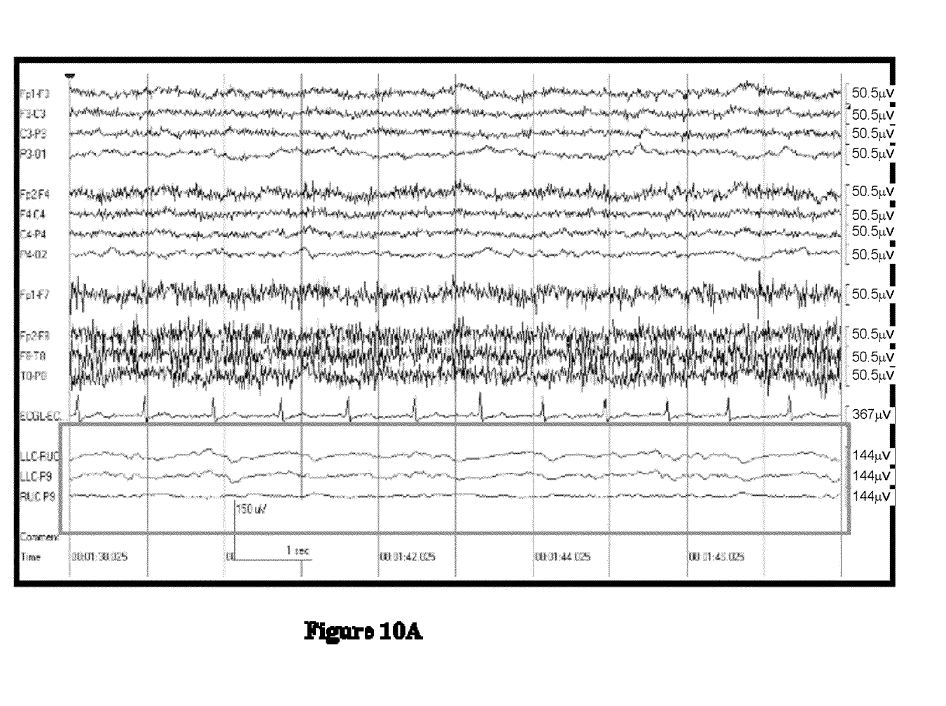

FIGS. 9A-C show EEG recordings from a traditional scalp montage as well as from a TCME (boxed) and demonstrating seizure activity within TCME recordings without correlate in the overlying scalp coverage. Devices were inserted in a 76 year old woman with intraventricular hemorrhage (FIG. 9A), a 74 year old woman with Hunt/Hess grade III subarachnoid hemorrhage (FIG. 9B), and a 73 year old woman with Hunt/Hess grade III subarachnoid hemorrhage (FIG. 9C).

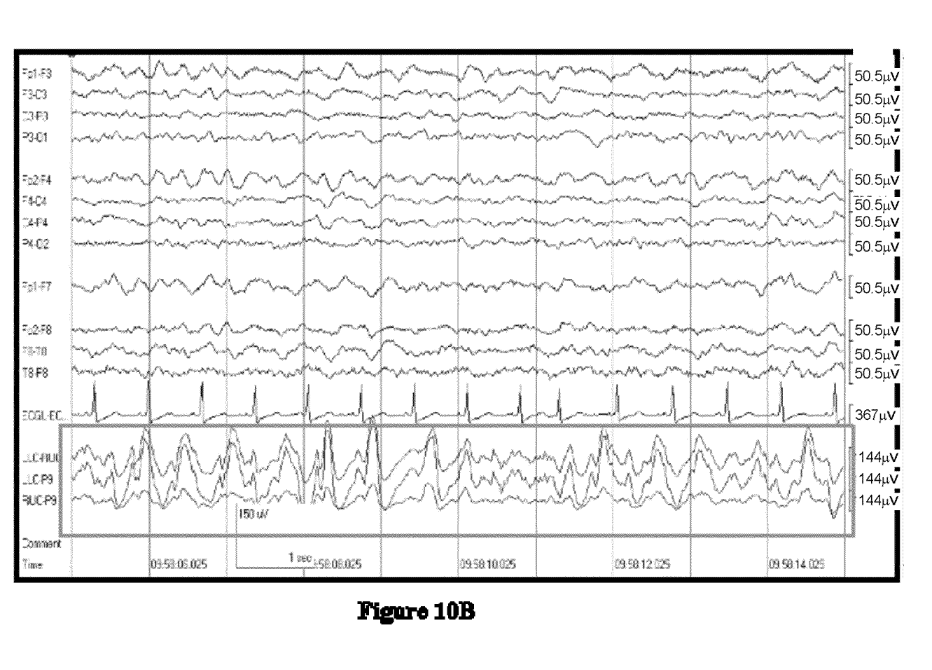

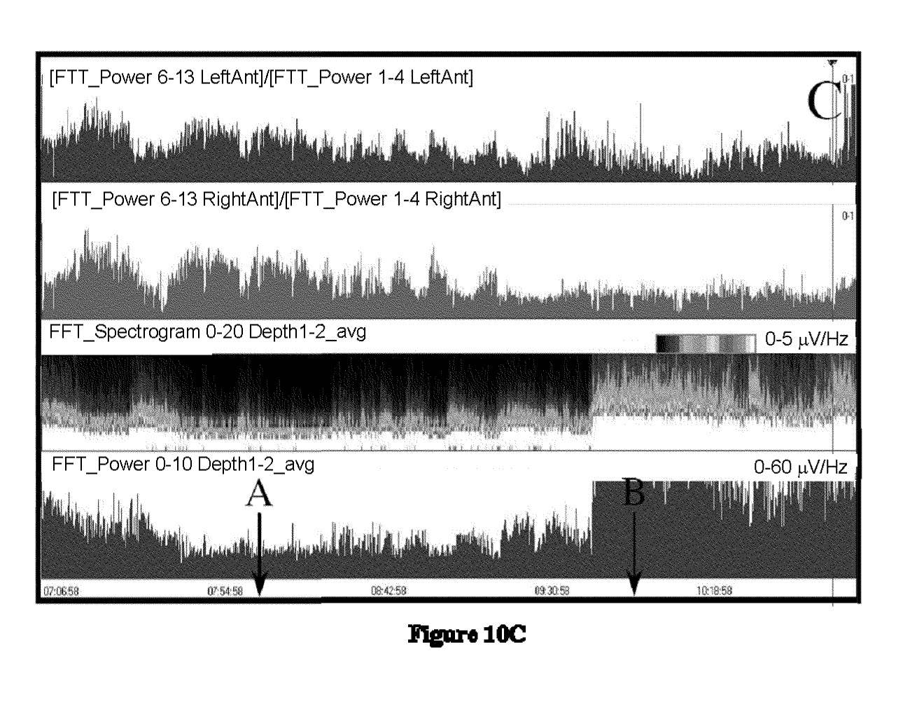

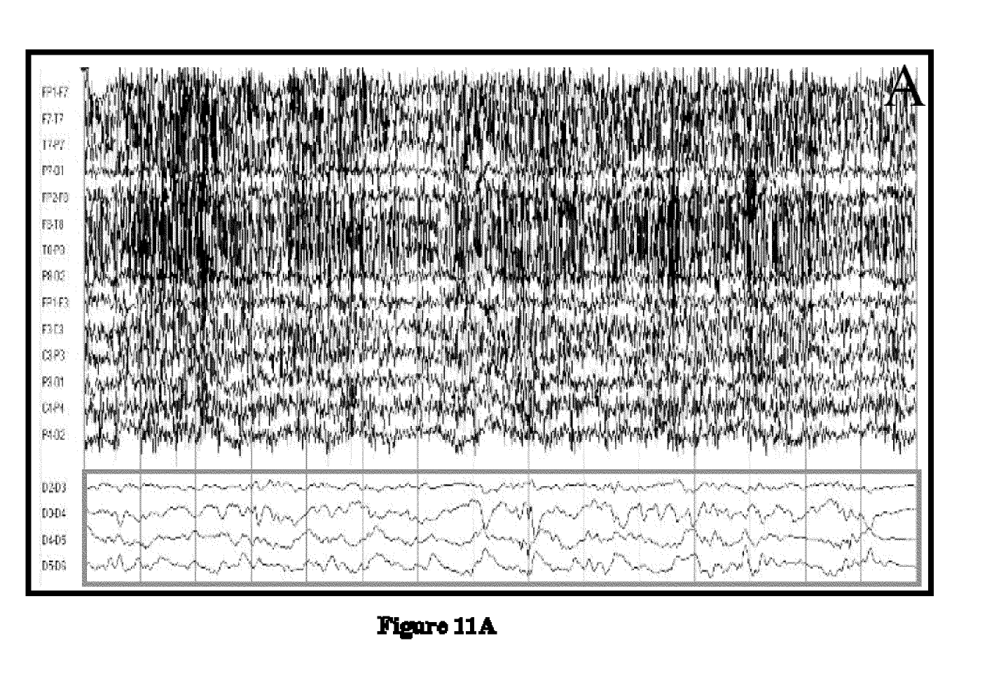

FIGS. 10A-C show selected EEG and TCME recordings and CSA analysis from a 80 year old woman with Hunt/Hess grade III subarachnoid hemorrhage and demonstrate the rapid development of seizures detected in isolation by a TCME (boxed). FIG. 10A shows baseline EEG activity. FIG. 10B shows seizure activity evident in the TCME without scalp electrode correlate. FIG. 10C shows CSA analysis demonstrating a sudden increase in EEG power in the TCME alone, associated with the onset of seizure activity visualized in raw EEG recordings.

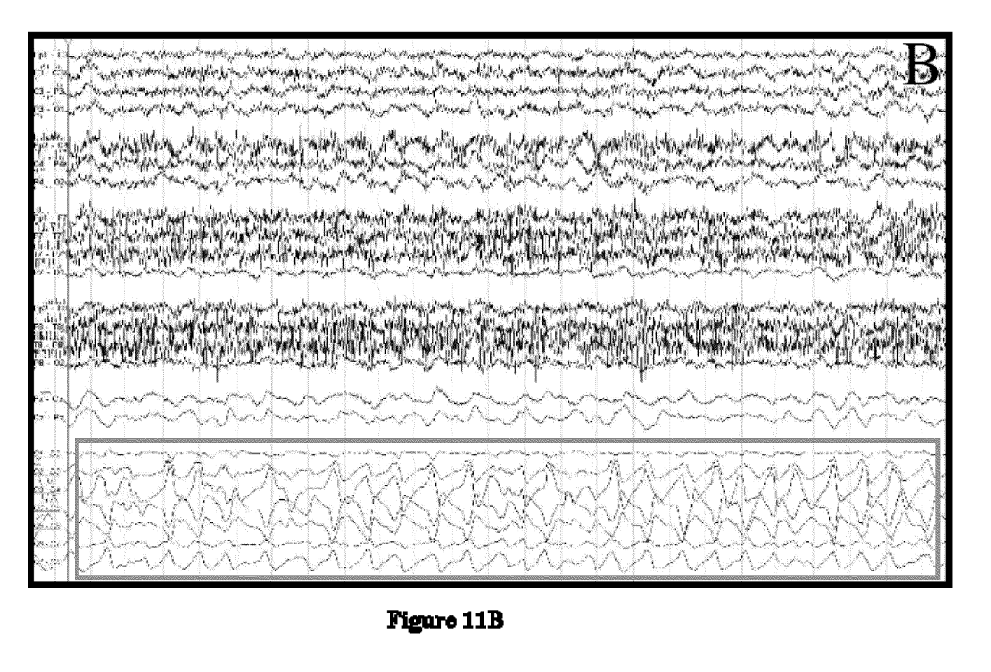

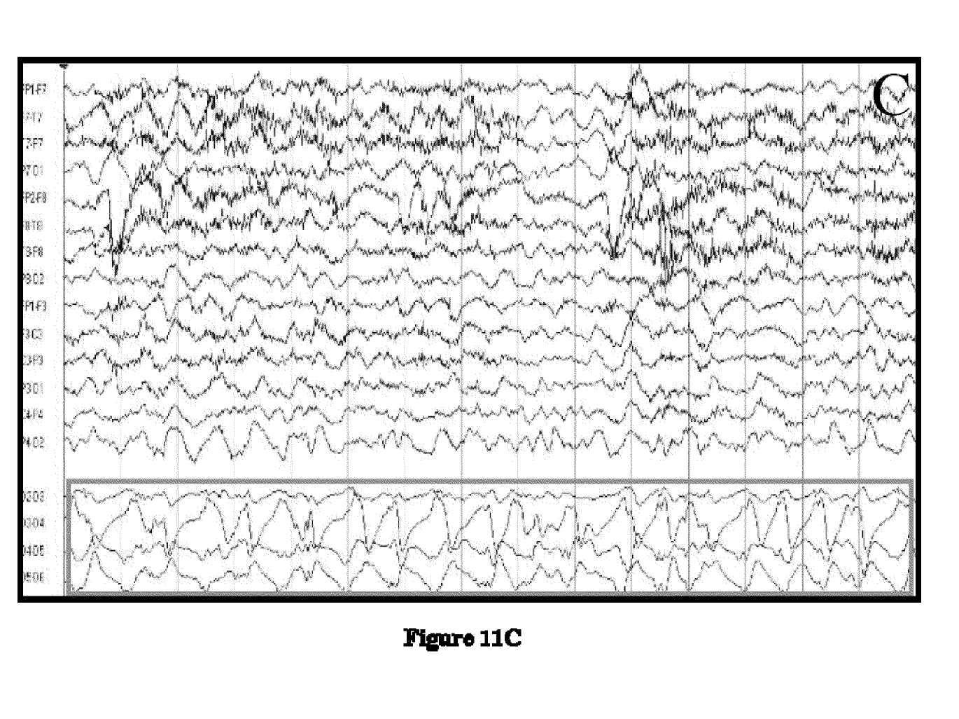

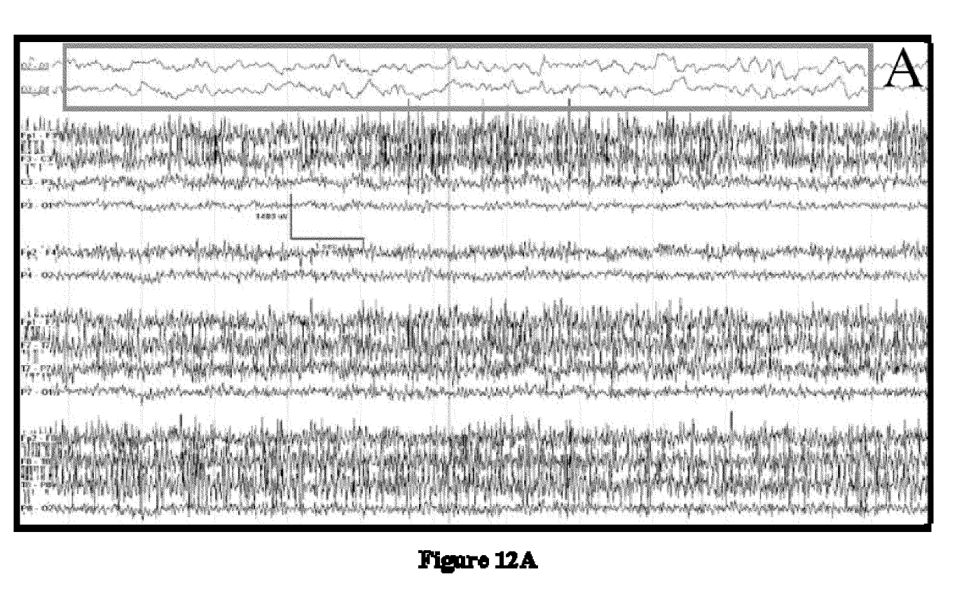

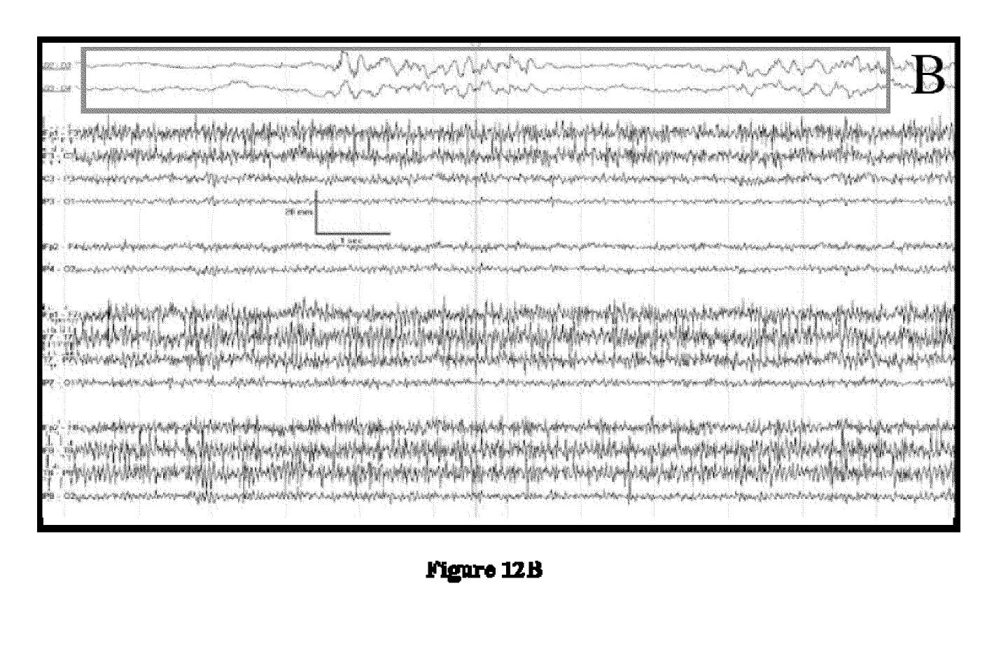

FIGS. 11A-C show EEG and TCME tracings of seizure activity in a 20 year old woman who suffered traumatic brain injury. FIGS. 11A and 11B show TCME tracings (boxed) demonstrating the development of seizure activity on day seven post implantation. Scalp tracings are significantly contaminated with myogenic artifact secondary to cooling in FIGS. 11 and 11B. FIG. 11C shows a period of improved scalp artifact that shows intermittent potential correlation with peak potentials seen in TCME derived recordings.

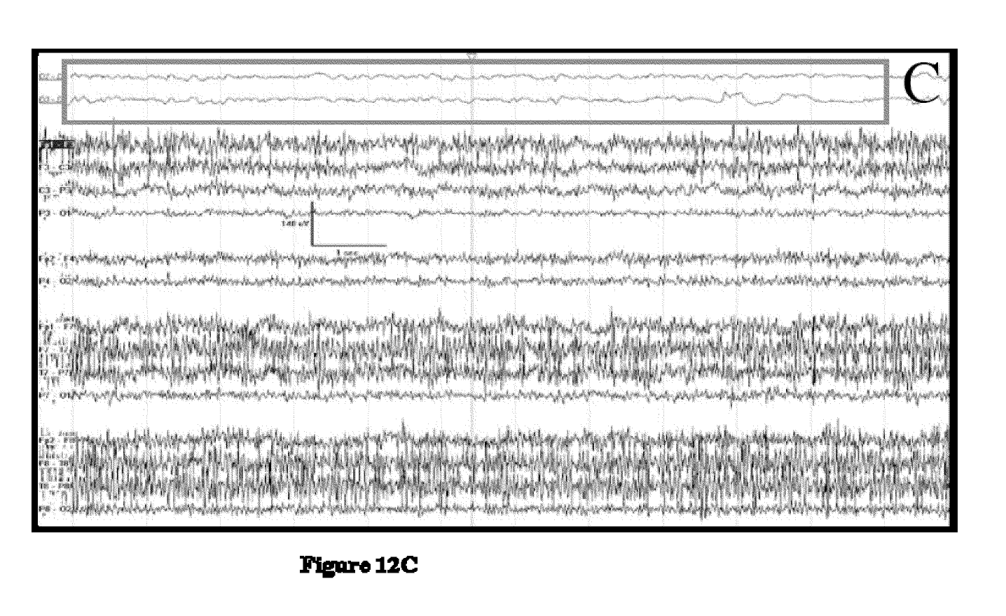

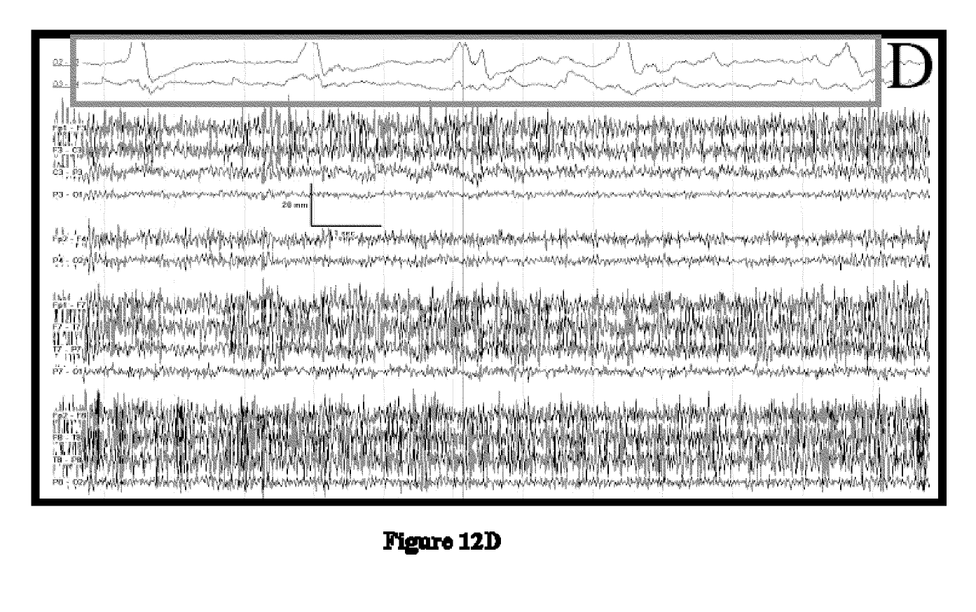

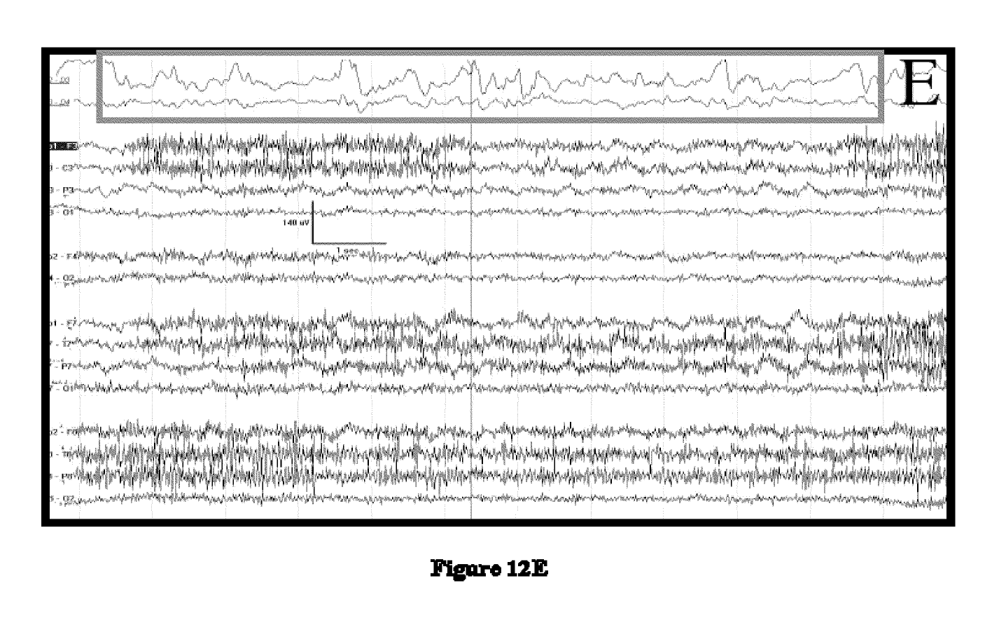

FIGS. 12A-E show data recorded from a patient with acute neurophysiological changes secondary to hemorrhagic conversion of a large right MCA infarction. The patient did not exhibit a significant change in neurological exam until 6-9 hours after the detection of EEG changes. FIG. 12A provides baseline EEG data at 9 pm (TCME data outlined); scalp EEG is essentially non-interpretable secondary to muscle artifact from shivering. FIG. 12B demonstrates a sudden conversion to a burst-suppression pattern at 9:45 pm recorded in isolation by the TCME with no clear change in the limited scalp EEG. FIG. 12C reveals near flattening of local potentials by 4 am. By 6:30 am, periodic delta waves reappeared (FIG. 12D). Continuous background EEG reappeared by 7:30 am (FIG. 12E). Time-locked data from the other neuromonitoring devices including ICP monitor, Licox monitor, and microdialysis catheter is provided in FIG. 5E. Although a transient spike in ICP is demonstrated, intracranial pressure rapidly returns to baseline levels. No significant change in lactate/pyruvate level was detected by microdialysis (already markedly elevated prior to event). Although brain oxygenation decreased, as detected by the Licox monitor, the drop was not detected until 2 hours after changes seen in the EEG recorded from the TCME.

Discussion

Considerable variation exists amongst EEG patterns associated with acute neurological injury, ranging from generalized seizure activity to diffuse slowing or attenuation. Notably, the clinical phenomenon of coma can be attributed to decreased activity of brain tissue (secondary to limited blood flow, increased pressure, etc.) or, paradoxically, neuronal hyperexcitability (as is seen in non-convulsive status epilepticus). The use of EEG in the ICU setting has a central role in the monitoring of comatose patients. However, limitations of traditional scalp EEG have hampered further development of this technique as a component of effective neurophysiological monitoring.

The results described herein describe a method for performing intracranial EEG recording in patients with critical neurological injuries, utilizing bedside insertion of a transcortical “mini-depth” multicontact electrode. The results described herein further demonstrate that clinical use of TCME is safe and provides high quality data in the ICU setting. Improvements in signal to noise ratio were observed from TCME when compared to concurrently recorded scalp EEG. The majority of the patients examined displayed seizures or periodic epileptiform discharges in TCME recordings that were not detectable in scalp EEG. These results showed the insensitivity of surface electrodes in detecting focal EEG changes that can signify or contribute to ongoing neuronal injury. In addition, sudden event-related, TCME-specific EEG changes were observed in two patients who suffered secondary non-seizure neurological complications. These changes anticipated event detection by other monitoring modalities (including scalp EEG) by at least several hours, emphasizing the potential clinical utility of TCME as a component of continuous, real-time neurophysiological monitoring.

The significance of epileptiform EEG abnormalities recorded by the TCME is currently unclear, particularly when these changes are detected exclusively by TCME (not by the overlying scalp electrodes). Focal EEG changes recorded by the TCME in the cohort may serve as indicators for membrane instability or metabolic stress induced (or worsened) by neurological injury. As the monitored cortex was structurally normal at the site of device insertion in the majority of the patients, observed TCME-specific EEG abnormalities can reflect effects of diffuse neuronal irritation (e.g., inflammation from subarachnoid blood), tenuous metabolic support (e.g., decreased cerebral blood flow and oxygen delivery), or uncoupling of central control elements of cortical firing patterns (i.e., disruption of thalamocortical circuitry). In some cases, the identification of these changes may allow for targeted therapeutic intervention and subsequent real-time monitoring of appropriate physiological responses to these interventions. High-fidelity focal monitoring, as can be performed with the TCME, will also play an important role in the further evaluation of a hypothetical “ictal-interictal” continuum in patients with acute neurological disease, in which injured brain tissue exists in a pre-epileptogenic state serving as a prelude to increased synchronization and generalized epileptiform activity. The presence of focal cortical seizures may also be clinically relevant as a treatable source of increased metabolic demand, most relevant in brain tissue rendered susceptible by prior injury, and therefore mandate therapeutic intervention to attempt resolution of the abnormal activity. Ongoing analysis of concurrently recorded microdialysis data from these patients will shed light on the potential association between localized seizure activity and increased metabolic stress).

A state of diffuse neuronal hyperexcitability exists after a focal neurological insult (Mun-Bryce et al, 2004). Intracranial recording in patients with neurological injury have also demonstrated that abnormal electrophysiological discharges (including cortical spreading depression and peri-infarct depolarizations) can be detected that are not seen in surface EEG recordings (Strong et al, 2002; Fabricius et al, 2006; Dohmen et al, 2008). These discharges may contribute to neurological injury (Fabricius et al, 2006). This result is supported by data from animal models of transient focal ischemia and reperfusion, which indicate that similar peri-infarct depolarizations contribute to secondary brain injury via delayed edema, intracranial hypertension, and cerebral hypoperfusion (Hartings et al, 2006). Some clinical data shows that electrographic seizures recorded with surface EEG in patients with non-traumatic ICH are associated with increasing mass effect, midline shift, expanding hemorrhage and worse clinical outcomes (Vespa et al, 2003; Claassen et al, 2007).

“Miniseizure” activity detected by TCME can be representative of the abnormal, yet asynchronous, activity of a large number of diffuse, independent cortical generators variously affected in the course of acute brain injury which fail to reach a level of synchronization allowing for detection by scalp EEG. These miniseizures may in fact underlie surface EEG findings that fall within the “ictal-interictal continuum” (Chong et al, 2005). Furthermore, it is possible that multifocal, asynchronous miniseizures contribute to global cortical dysfunction and encephalopathy frequently observed in patients with neurological injuries, potentially leading to persistent coma or delayed recovery of neurological function.

The detection of focal cortical seizures can have additional clinical relevance as a potentially treatable source of increased metabolic demand in brain tissue rendered susceptible to metabolic insufficiency by prior injury. Measured elevations of biomarkers for neuronal injury (such as neuron-specific enolase, glycerol, glutamate, and increased lactate-pyruvate ratio) indicate that seizures can cause neuronal damage (Vespa et al, 2007; Henshall and Murphy 2008; Schreiber et al, 1999). In several individuals from the results described herein, seizures detected by TCME were associated with an increase in the local lactate to pyruvate ratio, determined via microdialysis catheter sampling. Comparative analysis of data from the subset of patients with microdialysis catheters undergoing TCME recording is currently being performed on time-locked episodes of abnormal EEG activity to identify concurrent changes in lactate/pyruvate levels that can indicate local metabolic stress.

Although the volume of cortex accessible to recording by a single transcortical electrode is limited, detailed anatomic targeting will not be necessary in the majority of cases. Rather, regionally chosen cortex (selected for safety and procedural convenience) will function as the “representative voice” for a relatively larger, yet physiologically linked, volume of brain tissue. In this regard, the TCME parallels current invasive neuromonitoring systems (e.g., ICP, Licox, and microdialysis monitors) to provide physiologic data for large regions of cortex. Notably, TCME recordings from the two patients who suffered profound secondary neurological complications indicates that specific targeting of the electrode insertion site is not critical in cases where catastrophic or globally relevant changes have occurred. In these cases, relevant intracranial events were rapidly and clearly identified by TCME recordings prior to the detection of changes in neurological exam, scalp EEG or data from other implanted monitoring devices. Early detection and therapeutic intervention, particularly in the setting of worsening cerebral ischemia, makes TCME an attractive option for further inclusion in neuromonitoring systems. The improved data quality from TCME recordings can be instrumental in the continued development of real-time “neurotelemetry” and automated EEG-based alarm systems.

Although insertion-related cortical injury and subsequent seizure activity can occur with the use of depth electrodes, injury potentials in animal models and surgical epilepsy patients are self-limited (Ulbert et al, 2004). In addition, interpretation of the clinical relevance of EEG abnormalities seen in isolated, focal recordings from these patients may not parallel conclusions drawn from previous studies using scalp-based EEG. Data acquired in a parallel fashion from other physiological monitors can provide information regarding the metabolic impact of electrographic patterns detected by intracortical electrodes, as well as the basic physiological determinants of this abnormal activity. The independent technical aspects involved with the bedside insertion of currently available neuromonitoring devices mandate the participation of experienced personnel in order to achieve reliable and reproducible results.

Expertise can be necessary for the optimization of TCME recording, and comparisons of EEG recorded by TCME. Further, scalp electrodes can be time consuming and expensive. As patients are continuously monitored, the need for twenty-four hour review to guide therapeutic responses requires considerable manpower at the current time. These factors provide increased impetus for the development of automated EEG analysis platforms which, when combined with high-fidelity data from TCME recordings, will allow for the optimization of bedside alarm systems. Such technology can be central in the monitoring, treatment, and potentially improved outcomes of patients with critical neurological injuries.

### Example 2

### Comparative Example

A device utilizing intracranial electrodes in combination with a ventricular drain has previously been described (see Karasawa et al., Clin. Neurophysiol. 2001 January; 112(1):25-30). The device described by Karasawa et al. is designed for placement in subjects undergoing emergent neurosurgical procedures. The placement of the electrodes on the device described in Karasawa et al. is such that insertion of the device into the brain of a subject will cause one electrode to contact in the ventricle of the brain, one electrode to contact the white matter of the brain and one electrode to contact the subdural space of the brain. This type of configuration will result in the recorded potentials represented as the averaged/summed signals generated by large volumes of brain tissue rather than focal cortical potentials.

Karasawa et al. describe that potentials of identical amplitude were recorded from all contacts, including the intraventricular electrode, of the device Because the design limited the number of potential intracortical contacts to one, the potentials recorded in Karasawa et al. represent averaged/summed signals generated by large volumes of brain tissue rather than focal cortical potentials. The device described in Karasawa is not suitable for use in bedside transcortical monitoring of subjects with acute neurological injury.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the subject matter disclosed herein.
Vespa P M. Multimodality monitoring and telemonitoring in neurocritical care: from microdialysis to robotic telepresence. Curr Opin Crit Care 2005; 11: 133-138.
Towne A R. Prevalence of nonconvulsive status epilepticus in comatose patients. Neurology 2000; 54: 340-345.
Claassen J, Mayer S A, Kowalski R G, et al. Detection of electrographic seizures with continuous EEG monitoring in critically ill patients. Neurology 2004; 62: 1743-1749.
Vespa P M, Miller C, McArthur D, et al. Nonconvulsive electrographic seizures after traumatic brain injury result in a delayed, prolonged increase in intracranial pressure and metabolic crisis. Crit Care Med 2007; 35: 2830-2836.
Sundt T M Jr, Sharbrough F W, Anderson R E, et al. Cerebral blood flow measurements and electroencephalograms during carotid endarterectomy. J Neurosurg 1974; 41: 310-320.
Vespa P M, Nuwer M R, Juhasz C, et al. Early detection of vasospasm after acute subarachnoid hemorrhage using continuous EEG ICU monitoring. Electroencephalogr Clin Neurophysiol 1997; 103: 607-615.
Claassen J, Hirsch L J, Kreiter K T, et al. Quantitative continuous EEG for detecting delayed cerebral ischemia in patients with poor-grade subarachnoid hemorrhage. Clin Neurophysiol 2004; 115: 2699-2710.
Young G B, Campbell V C. EEG monitoring in the intensive care unit: pitfalls and caveats. J Clin Neurophysiol 1999; 16: 40-45.
Mun-Bryce S, Roberts L J, Hunt W C, et al. Acute changes in cortical excitability in the cortex contralateral to focal intracerebral hemorrhage in the swine. Brain Res 2004; 12: 218-226.
Strong A J, Fabricious M, Boutelle M G, et al. Spreading and synchronous depressions of cortical activity in acutely injured human brain. Stroke 2002; 33: 2738-2743.
Fabricius M, Fuhr S, Bhatia R, et al. Cortical spreading depression and peri-infarct depolarization in acutely injured human cerebral cortex. Brain 2006; 129: 778-790.
Dohmen C, Sakowitz O W, Fabricious M, et al. Spreading depolarizations occur in human ischemic stroke with high incidence. Ann Neurol 2008; 63: 720-728.
Hartings J A, Tortella F C, Rolli M L. AC electrocorticographic correlates of peri-infarct depolarizations during transient focal ischemia and reperfusion. J Cereb Blood Flow Metab 2006; 26: 696-707.
Vespa P M, O'Phelan K, Shah M, et al. Acute seizures after intracerebral hemorrhage: a factor in progressive midline shift and outcome. Neurology 2003; 60: 1441-1446.
Claassen J, Jette N, Chum F, et al. Electrographic seizures and periodic discharges after intracerebral hemorrhage. Neurology 2007; 69: 1356-1365.
Chong D J, Hirsch L J. Which EEG patterns warrant treatment in the critically ill? Reviewing the evidence for treatment of periodic epileptiform discharges and related patterns. J Clin Neurophysiol 2005; 22: 79-91.
Henshall D C, Murphy B M. Modulators of neuronal cell death in epilepsy. Curr Opin Pharmacol 2008; 8: 75-81.
Schreiber S S, Sun N, Tocco G, et al. Expression of neuron-specific enolase in adult rat brain following status epilepticus. Exp Neurol 1999; 159: 329-331.
DeGiorgio C M, Correale J D, Gott P S, et al. Serum neuron-specific enolase in human status epilepticus. Neurology 1995; 45: 1134-1137.
Ulbert I, Magloczky Z, Eross L, et al. In vivo laminar electrophysiology co-registered with histology in the hippocampus of patients with temporal lobe epilepsy. Exp Neurol 2004; 187: 310-318.

1. A device for measuring intracortical brain activity of a subject, comprising:
(a) a support member comprising at least a proximal end, a distal end, and an electrode region, wherein the support member is configured for intracranial insertion in a subject;
(b) a plurality of electrodes positioned in the electrode region of the support member, wherein the electrode region is configured to traverse the cerebral cortex of the subject when the support member is inserted intracranially in the subject such that two or more electrodes in the electrode region contact the cerebral cortex of the subject, and
(c) a plurality of conductors, each conductor individually connected to a corresponding electrode of the plurality of electrodes.

2. A device for measuring intracortical brain activity of a subject, comprising:
(a) a hollow support member having an interior surface and an exterior surface, the support member comprising at least a proximal end, a distal end, an electrode region and a drainage region, wherein the support member is configured for intracranial insertion in a subject;
(b) one or more drainage holes in the drainage region, wherein the drainage holes provide fluid contact between the interior surface of the support member and the exterior surface of the support member,
(c) a plurality of electrodes positioned in the electrode region of the support member, wherein the electrode region and the drainage region are positioned along the support member such that two or more electrodes in the electrode region contact the cerebral cortex of the subject when the drainage region of the support member is in fluid contact with cerebrospinal fluid in the brain ventricle of the subject, and
(d) a plurality of conductors, each conductor individually connected to a corresponding electrode of the plurality of electrodes.

3. The device of claim 1 or 2, wherein the conductors are electrically coupled to an external sensing unit.

4. The device of claim 1 or 2, wherein the conductors extend through the interior of the support member from each electrode to an external sensing unit.

5. The device of claim 1 or 2, wherein the conductors extend outside of the support member from each electrode to the external sensing unit.

6. The device of claim 3, wherein the sensing unit is an electroencephalograph (EEG) sensing unit.

7. The device of claim 3, wherein the sensing unit is an EEG amplification and recording system.

8. The device of claim 1 or 2, wherein the support member has a substantially tubular cross section.

9. The device of claim 1 or 2, wherein the support member comprises silicone.

10. The device of claim 1 or 2, wherein the electrode region is positioned from about 3.5 cm to about 0.1 cm from the distal end of the support member.

11. The device of claim 1 or 2, wherein the electrode region spans a dimension of about 1.9 cm along the length of the support member.

12. The device of claim 2, wherein the electrode region is positioned about 2 cm from the drainage region.

13. The device of claim 1 or 2, where the device comprises at least 2 electrodes in the electrode region, at least 3 electrodes in the electrode region, at least 4 electrodes in the electrode region, at least 5 electrodes in the electrode region, at least 6 electrodes in the electrode region, at least 7 electrodes in the electrode region, or at least 8 electrodes in the electrode region.

14. The device of claim 1 or 2, wherein the device comprises 8 electrodes in the electrode region.

15. The device of claim 1 or 2, wherein at least one electrode in the electrode region contacts a brain region other than the cerebral cortex when the device is inserted intracranially in the subject.

16. The device of claim 1 or 2, wherein two or more of the electrodes in the electrode region are at about 0.1 mm to about 5 mm in width along the length of the support member.

17. The device of claim 1 or 2, wherein two or more of the electrodes in the electrode region are at about 1 mm in width along the length of the support member.

18. The device of claim 1 or 2, wherein the electrodes in the electrode region are spaced such that the inter-electrode distance from the center of one electrode to the center of an adjacent electrode is about 0.1 mm to about 5.0 mm.

19. The device of claim 1 or 2, wherein the electrodes in the electrode region are spaced such that the inter-electrode distance from the center of one electrode to the center of an adjacent electrode is 2 mm.

20. The device of claim 1 or 2, wherein the device further comprises one or more probes for detecting an additional brain parameter.

21. The device of claim 20, wherein the probe is positioned on the support member and configured to contact the brain of the subject when the device is inserted intracranially.

22. The device of claim 20, wherein the probe is a cerebral blood flow probe, a thermal diffusion probe, a oxygen sensing probe, a catheter or any combination thereof.

23. The device of claim 22, wherein the catheter is a microdialysis catheter.

24. The device of claim 20, further comprising one or more conductors are individually connected to each probe and extends from probe to a recording system outside of the support member.

25. A method for measuring abnormal electrical activity in the cerebral cortex of a subject, the method comprising:
(a) inserting the device of claim 1 into the brain of the subject such that two or more of electrodes in the electrode region of the device are in physical contact with the cerebral cortex of the subject;
(b) measuring the electrical activity values recorded from the two or more electrodes in the electrode region of the device in physical contact with the cerebral cortex of the subject; and
(c) performing bi-polar referencing of the electrical activity values recorded from the two or more electrodes in the electrode region of the device in physical contact with the cerebral cortex of the subject.

26. A method for measuring abnormal electrical activity in the cerebral cortex of a subject, the method comprising:
(a) inserting the device of claim 2 into the brain of the subject such that the drainage region of the device is in fluid contact with cerebrospinal fluid of a brain ventricle of the subject and two or more of electrodes in the electrode region of the device are in physical contact with the cerebral cortex of the subject;
(b) measuring the electrical activity values recorded from the two or more electrodes in the electrode region of the device in physical contact with the cerebral cortex of the subject; and
(c) performing bi-polar referencing of the electrical activity values recorded from the two or more electrodes in the electrode region of the device in physical contact with the cerebral cortex of the subject.

27. The method of claim 25 or 26, wherein the abnormal brain activity is an electrographic seizure, a periodic epileptiform discharge, suppression-burst activity, spontaneous variability, reactivity to external stimuli, a presence of stage II sleep transients, a state change, a stimulus-induced rhythmic, periodic or ictal discharges (SIRPIDs), or any combination thereof.

28. A bedside alarm system comprising
(a) a monitor adapted to receive at least electroencephalogram signals as an input from the device of claim 1, 2 or 20 and to produce an output of numerical or graphical values indicative of intracortical brain activity of a subject; and
(b) a signal processor connected to receive the output indicative of intracortical brain activity of the subject and programmed to analyze that output to detect a change in brain function; and
(c) a feedback transducer connected to the signal processor so as to selectively produce an alarm signal to the subject in the event the signal processor detects onset of the change in brain function.

29. The bedside alarm system of claim 28, wherein the electroencephalogram signal is a continuous electroencephalogram signal.

30. The bedside alarm system of claim 28, wherein the change of brain function is a seizure in the subject, a change in blood flow in the brain of the subject, a change in intracranial pressure, or any combination thereof.
